(19) United States
(12) Patent Application Publication
(10) Pub. No.: US 2015/0352246 A1
(43) Pub. Date: Dec. 10, 2015

(54) SEALANTS HAVING CONTROLLED DEGRATION

(71) Applicant: PROLYNX LLC, San Francisco, CA (US)

(72) Inventors: Jeffrey C. HENISE, San Francisco, CA (US); Gary W. ASHLEY, Alameda, CA (US); Daniel V. SANTI, San Francisco, CA (US)

(73) Assignee: Prolynx LLC, San Francisco, CA (US)

(21) Appl. No.: 14/762,424

(22) PCT Filed: Jan. 22, 2014
(86) PCT No.: PCT/US14/12571

§ 371 Date: Jul. 21, 2015

Related U.S. Application Data
(60) Provisional application No. 61/755,405, filed on Jan. 22, 2013.
(60) Provisional application No. 61/774,498, filed on Mar. 7, 2013.

(51) Int. Cl. A61L 26/00; A61L 24/00; A61L 24/06

(52) U.S. Cl. CPC A61L 26/0019; A61L 26/008; A61L 26/009; A61L 26/0066; A61L 24/06; A61L 24/0015; A61L 24/0031; A61L 24/0042; A61L 26/0085; A61L 24/0036; A61L 2300/604; A61L 2300/216

(57) ABSTRACT

The invention provides sealants wherein biodegradable hydrogels that do not otherwise comprise protein-reactive groups for binding to membranes or tissue are provided said groups optionally through a linker. The linker may be biodegradable and may be biodegradable by an elimination reaction. The invention also provides multilayer gels for drug delivery wherein a porous gel in contact with a tissue or organ to which the drug is to be delivered is protected by a microporous layer from the surrounding bodily fluid.

### RELATED APPLICATIONS

This application claims benefit of U.S. application Ser. No. 61/755,405 filed 22 Jan. 2013 and U.S. application Ser. No. 61/774,498 filed 7 Mar. 2013 which are incorporated herein by reference in their entirety.

### TECHNICAL FIELD

Polymeric surgical sealants are used to provide leak-free closures around sutures and surgical anastomoses. Such sealants must have adequate tissue adherence and sufficient mechanical strength to withstand fluid pressure from the suture needle holes, and must be sufficiently flexible to maintain integrity and continue sealing during the post-surgery recovery process. Polymerization should be sufficiently rapid to allow quick wound closure during surgery. After recovery is complete and the suture wound has healed, the sealant should degrade and be reabsorbed. Sealants are also used as adhesion barriers, films, fabrics, gels and other materials that are applied between layers of tissues at the end of a surgery. An adhesion barrier acts as a physical barrier to separate tissue surfaces so that they do not adhere to one another while the tissue surfaces heal. The adhesion barrier should dissolve and be absorbed by the body after the healing process is complete. Other sealants are used as tissue adhesives for sutureless closure of wounds (Mizrahi, et al., “Tissue Adhesives as Active Implants,” in Active Implants and Scaffolds for Tissue Regeneration, M. Zilbermann, Ed., Springer, 2011, pp 39-56).

### BACKGROUND ART

A number of sealants are currently marketed, including DuraSeal® (Confluent Surgical, Waltham, Mass.; Covidien), a 4-arm 20-kDa polyethylene glycol crosslinked with trilysine, used to prevent leakage of cerebrospinal fluid from dural sutures during spinal surgery; it is hydrolyzed and absorbed over a 4-8 week period. A newer formulation using a lower molecular weight polyethylene glycol, DuraSeal® Exact, has been reported to provide a tighter hydrogel matrix with less swelling than the original formulation. It is degraded by hydrolysis and reabsorbed over a 9-12 week period. In both cases, the hydrogel is thought to adhere to tissue by mechanical means. CoSeal® (Angiotech Pharmaceuticals, Vancouver, BC; Baxter), a mixture of a 4-arm PEG tetra-hydroxysuccinimide ester and a 4-arm PEG tetra thiol, each of approximate MW 10 kDa, used for arterial and vascular reconstruction. The resulting gel comprises thioester linkages that are hydrolytically labile, resulting in eventual gel degradation and resorption. Tissue adherence is provided by reaction of some of the reactive hydroxysuccinimide esters, and possibly some of the thioester groups, with protein amine groups in the tissue. CoSeal® is reported to remain effective at the application site for 7 days, and is fully degraded after 30 days. Hemaseel® (Haemacure Corporation, Montreal, Calif.), a fibrin-based sealant used between skin grafts and wound sites. The use of the fibrin sealant between the skin graft and the wound bed interface provides adhesive qualities allowing fixation of the graft without the use of staples or sutures and seals the tissue bed layer, thereby inhibiting seroma or hematoma formation without compromising the healing process, resulting in a higher percentage of graft take with a more acceptable cosmetic outcome than using mechanical fixation. Omnex® (Ethicon, Somerville, N.J.), a mixture of 2-octyl cyanoacrylate and butyl lactoyl cyanoacrylate, used in vascular reconstructions. Omnex® degrades by hydrolysis over approximately 36 months. While cyanoacrylates have also been used as tissue adhesives, for example DermaBond® (Omnex®), their use is limited by toxicity, such as tissue necrosis at the site of application. Progel® (Neomend, Irvine, Calif.), human serum albumin crosslinked with a bifunctional hydroxysuccinimidyl-polyethylene glycol (U.S. Pat. No. 6,899,889 B1), used for intraoperative sealing of pleural air leaks. A formulation using a recombinant albumin, Progel® Platinum Surgical Sealant, has been developed. Progel® AB is a hydrogel adhesion barrier sealant that can be sprayed onto general visceral organs during surgery to help prevent post-operative adhesions. Approximately 60% of Progel® is degraded after 1 day, and complete degradation is observed after 2 weeks. BioGlue® (CryoLife, Kennesaw, Ga.) is a mixture of albumin (supplied as a 45% solution) and glutaraldehyde (supplied as a 10% solution) used in cardiovascular surgery including arteriovenous access, aortobifemoral bypass, femoral popliteal bypass, endarterectomy, abdominal aortic aneurysm and aortotomies. Toxicity has been reported due to released glutaraldehyde (Fuerst & Banerjee, “Release of Glutaraldehyde From an Albumin-Glutaraldehyde Tissue Adhesive Causes Significant In Vitro and In Vivo Toxicity,” Ann. Thoracic Surg (2005) 79:1522-1528), and the stiffness of the polymerized material may cause mechanical issues with flexible tissues. FocalSeal-L® (Genzyme, Cambridge, Mass.) is a mixture of a polyethylene glycol capped with short segments of acrylate-capped poly(L-lactide) and poly(trimethylene carbonate) with a photoinitiator, eosin Y, and has been used to limit air leak after pulmonary resection. The solution polymerizes upon exposure to blue-green light to form a thin film hydrogel. The sealant does not bond covalently with tissue, and expands upon contact with bodily fluids over approximately 24 hours. Hydrolysis of the lactide and carbonate linkages allows for gel degradation and resorption. FocalSeal® has been used as a tissue adhesive. Adherus® Dural Sealant and Spinal Sealant (HyperBranch Medical Technology, Durham, N.C.), a mixture of poly(ethylene imine) crosslinked with a bifunctional PEG-hydroxy-succinimidyl ester, used in cranial and spinal surgery to prevent cerebrospinal fluid leakage and dural adhesions. OcuSeal® Liquid Ocular Bandage (HyperBranch Medical Technology, Durham, N.C.), a synthetic hydrogel that is applied directly to the ocular surface as a liquid, using a brush applicator.

U.S. Pat. No. 7,151,135 (issued 19 Dec. 2006) and U.S. Pat. No. 7,176,256 (issued 13 Feb. 2007) disclose crosslinked synthetic polymers for use as bioadhesives among other uses. U.S. Pat. No. 8,303,973 (issued 6 Nov. 2012) discloses sealants comprising PEG and chitosan. U.S. Pat. No. 6,602,952 (issued 5 Aug. 2003) discloses hydrogel sealants. U.S. Pat. No. 6,495,127 (issued 17 Dec, 2002) discloses sealants formed from multifunctional polymers together with a tensile strength enhancer. U.S. Pat. No. 6,458,147 (issued 1 Oct. 2002) discloses sealants formed from a protein such as albumin together with a polymer. U.S. Pat. No. 6,312,724 (issued 6 Nov. 2001) discloses sealants formed by reaction of a thiol-polymer and a thiol-reactive polymer.

Hydrogels offer several benefits for use as surgical sealants, such as high water content, tissue compatibility, good mechanical strength, and flexibility. A hydrogel is a 3-dimensional network of natural or synthetic hydrophilic polymer chains in which water (up to 99%) is the dispersion medium. Fragile macromolecules often require a well-hydrated environment for activity and structural integrity, and the high degree of hydration of a hydrogel may preserve the folding of a protein needed for its bioactivity. The high water content of the hydrogels render the material biocompatible and minimize inflammatory reactions of tissues in contact with the gel, and provide a flexibility comparable to that of living tissue. Hydrogels are thus of interest in biomedical engineering, as absorbent materials for wound dressings and disposable diapers, as carriers for extended drug release, and as flexible sealants for surgical procedures. Hydrogels have been prepared by physical or chemical crosslinking of hydrophilic natural or synthetic polymers. Examples of hydrogels formed from crosslinking of natural polymers include those formed from hyaluronans, chitosans, collagen, dextran, pectin, polylysine, gelatin or agarose. (See: W. E. Hennink and C. F. van Nostrum, Adv. Drug Del. Rev. (2002) 54:13-36; A. S. Hoffman, Adv. Drug Del. Rev. (2002) 43:3-12). These hydrogels consist of high-molecular weight polysaccharide or polypeptide chains. Some examples of their use include the encapsulation of recombinant human interleukin-2 in chemically crosslinked dextran-based hydrogels (J A. Cadee, et al., J. Control. Release (2002) 78:1-13) and insulin in an ionically crosslinked chitosan/hyaluronan complex (S. Surini, et al., J. Control. Release (2003) 90:291-301).

Examples of hydrogels formed by chemical or physical crosslinking of synthetic polymers include poly(lactic-co-glycolic)acid (PLGA) polymers, (meth)acrylate-oligolactide-PEO-oligolactide-(meth)acrylate, poly(ethylene glycol) (PEO), poly(propylene glycol) (PPO), PEO-PPO-PEO copolymers (Pluronie), poly(phosphazene), poly(methacrylates), poly(N-vinylpyrrolidone), PL(G)A- PEO-PL(G)A copolymers, poly(ethylene imine), and others. (See for example A. S Hoffman, Adv. Drug Del. Rev (2002) 43:3-12). Examples of protein-polymer encapsulation using such hydrogels include the encapsulation of insulin in physically crosslinked PEG-g-PLGA and PLGA-g-PEG copolymers (B. Jeong, et al., Biomacromolecules (2002) 3:865-868) and bovine serum albumin in chemically crosslinked acrylate-PGA-PEO-PGA-acrylate macromonomers (A. S. Sawhney, et al., Macromolecules (1993) 26:581-587). Hydrogels formed by crosslinking 4-arm PEGs have been disclosed (K. Nishi, et al., “Kinetic Study for AB-Type Coupling Reaction of Tetra-Arm Polymers,” Macromolecules (2012) 45:1031-1036; T. Sakai, et al., “Highly Elastic and Deformable Hydrogel Formed from Tetra-arm Polymers,” Macromolecular Rapid Comm. (2010) 31:1954-1959; T. Sakai, et al., “Design and Fabrication of a High-Strength Hydrogel with Ideally Homogeneous Network Structure from Tetrahedron-like Macromonomers,” Macromolecules (2008) 41:5379-5384; X. Li, et al., “Precise Control and Prediction of Hydrogel Degradation Behavior,” Macromolecules (2011) 44:3567-3571; T. Matsunaga, et al., “Structure Characterization of Tetra-PEG Gel by Small-Angle Neutron Scattering,” Macromolecules (2009) 42:1344-1351; T. Matsunaga, et al., “SANS and SLS Studies on Tetra-Arm PEG Gels in As-Prepared and Swollen States,” Macromolecules (2009) 42:6245-6252.

PCT application PCT/US2012/54278 and Ashley, et al., “Hydrogel Drug Delivery System with Predictable and Tunable Drug Release and Degradation Rates,” Proc. Natl. Acad. Sci. USA (2013) 110:2318-2323) describe degradable hydrogels having precisely controlled rates of degradation and optionally having controlled drug release. The gels described in the PNAS paper do not contain moieties that permit them to couple to proteins on membranes or cells for which sealants are desired. Neither do the exemplified hydrogels in the above cited PCT application. However, the generic class included in the PCT application would include instances where, by virtue of the functional groups employed to carry out the crosslinking, it is possible that some of the resulting hydrogels, due to having unreacted functional groups of particular types, would have the possibility to couple to proteins under appropriate conditions. For example, although the exemplified hydrogels form crosslinks by reaction between an azide and a cyclooctatriene moiety, neither of which will couple to protein, alternative possible functional groups may have this capacity. The present invention assures the presence of suitable functional groups on the surface of the hydrogel to effect linking to proteins.

### DISCLOSURE OF THE INVENTION

This invention provides degradable sealants having precisely controlled rates of degradation and optionally having controlled drug release and methods for their preparation and use. These sealants are expected to provide leak-free closures around sutures, surgical anastomoses, surgical implants, and wounds, as well as serve as adhesion barriers, tissue adhesives, and bandages. Controlled degradation rates are achieved through the use of beta-elimination linkers, which may be incorporated into the sealant crosslinks, drug attachment connectors, tissue attachment connectors, or combinations thereof.

In one aspect the invention provides sealants having controlled rates of degradation by virtue of crosslinkers that undergo β-elimination and optionally having controlled drug release. The sealants of the invention are crosslinked polymers wherein the crosslinks comprise groups that degrade by a pH-dependent elimination process thus allowing the sealant to be resorbed and wherein the sealants provide functional groups that promote tissue adherence. The protein-reactive functional groups that provide tissue adherence are linked to the polymer optionally via degradable linkers, and in some embodiments these linkers are biodegradable by elimination reactions. In some embodiments, the sealants of the invention further comprise drugs, wherein the drugs are covalently linked to the polymer optionally through linkers that degrade by a pH-dependent elimination process thereby releasing the drug.

In one aspect, the sealants of the invention comprise a biodegradable hydrogel coupled to a multiplicity of functional groups reactive with protein,

wherein said protein-reactive functional groups are coupled to the hydrogel through linkers of the formula

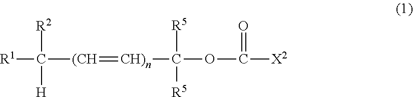

wherein

n is 0 or 1;

X² is a group that will allow attachment to the hydrogel or to protein;

at least one or both R¹ and R² is independently CN; NO₂;
optionally substituted aryl;
optionally substituted heteroaryl;
optionally substituted alkenyl;
optionally substituted alkynyl;
COR³ or SOR³ or SO₂R³ wherein
   R³ is H or optionally substituted alkyl;
   aryl or arylalkyl, each optionally substituted;
   heteroaryl or heteroarylalkyl, each optionally substituted; or
   OR⁹ or NR⁹₂ wherein each R is independently H or optionally substituted alkyl, or both R⁹ groups taken together with the nitrogen to which they are attached form a heterocyclic ring;
SR⁴ wherein
   R⁴ is optionally substituted alkyl;
   aryl or arylalkyl, each optionally substituted; or
   heteroaryl or heteroarylalkyl, each optionally substituted;

wherein R¹ and R² may be joined to form a 3-8 membered ring; and

wherein one and only one of R¹ and R² may be H or may be alkyl, arylalkyl or heteroarylalkyl, each optionally substituted; and

each R⁵ is independently H or is alkyl, alkenylalkyl, alkynylalkyl, (CH₂CH₂O)ₚ wherein p=1-1000, aryl, arylalkyl, heteroaryl or heteroarylalkyl, each optionally substituted; and

wherein at least one of R¹, R², and R⁵ is substituted with X² wherein one and only one of X² is a group that binds to the hydrogel and is not capable of binding to protein unless already coupled thereto and the other is part of a protein-reactive group, P. Thus, as shown, P would be represented by

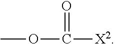

One example of formula (1) would then be

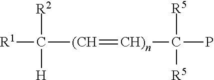

wherein P is OCOSu or OCO—NH—(CH₂)ₓ-R⁷; wherein R⁷ is maleimide, alpha-halocarbonyl, vinylsulfonyl, vinylsulfonamide, and the like; and wherein one R¹, R² or R⁵ can couple to hydrogel.

Alternatively, or in addition, the sealants comprise a biodegradable hydrogel coupled to a multiplicity of protein-reactive groups wherein the hydrogel comprises macromonomers which are coupled by crosslinkers of the formula

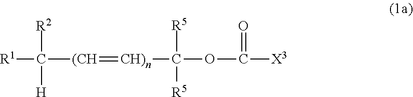

wherein

n is 0 or 1;

wherein one of R¹, R², and R⁵ is substituted with X³, wherein X³ is a functional group for binding the hydrogel and is not a protein-reactive group and R¹ , R² and R⁵ are otherwise as defined in formula (1) and/or

of the formula

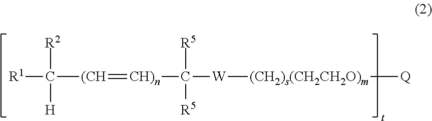

wherein one of R¹, R² and R⁵ in at least two of the t moieties shown within the bracket comprises said functional group X³ and R¹, R² and R⁵ are otherwise as defined in formula (1) and wherein

n is 0 or 1;

m is 0-1,000;

s is 0-2;

t is 2, 4, 8, 16 or 32,

W is O(C═O)O, O(C═O)NH, O(C═O), S,

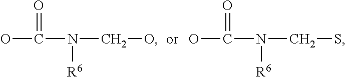

R⁶ is H, alkyl (1-6C), aryl or heteroaryl; and

Q is a core group having a valency=t.

In some embodiments, the sealants may also contain a releasable drug coupled through a biodegradable linker. In some embodiments, the linker is of formula (1b)

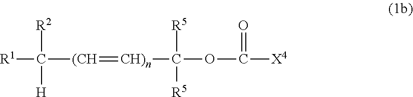

wherein n is 0 or 1 and one of R¹, R² and R⁵ is substituted with X⁴ wherein one X⁴ is a hydrogel binding group and the other is a drug binding group and neither X⁴ is a protein-reactive group unless already coupled to drug and wherein R¹, R² and R⁵ are otherwise as defined in formula (1).

Thus, in one example of formula (1b), this would result in

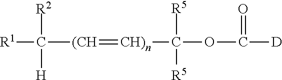

wherein D is a drug and one of R¹, R² and R⁵ can couple to hydrogel.

In some embodiments, the linked drug is of the formula

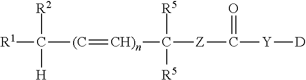

wherein n, R¹, R² and R⁵ are as defined above,

D is a residue of a drug or prodrug coupled through O, S or N;

Y is absent and Z is O or S; or

Y is NBCH₂ and Z is O;

wherein B is alkyl, aryl, arylalkyl, heteroaryl or heteroarylalkyl, each optionally substituted; and

wherein one of R¹, R² and R⁵ or B is coupled to the hydrogel.

Methods for preparation are also part of the invention and depend on the nature of the functional groups, the sequence of reaction of crosslinkers with the various components of the sealant, and the stoichiometry desired. Such methods are described in more detail below.

In another aspect the invention provides multi-layer hydrogels or sealants. In one embodiment, the multilayer hydrogels or sealants are especially useful for drug delivery to tissue that is normally in contact with a bodily fluid. In this embodiment, the multilayer hydrogel sealants for this purpose comprise a layer in contact with the tissue to which drug is to be delivered with sufficient porosity to deliver the drug to the tissue and is overlain with a layer with smaller pore size which prevents the contact of degradation enzymes from the fluid normally in contact with the tissue from contact with the drug in the drug delivery layer and, in some cases, can prevent the leakage of drug into the surrounding fluid.

In another aspect the invention provides methods for the use of the sealants of the invention and of the multilayer hydrogels or sealants.

### BRIEF DESCRIPTION OF THE DRAWINGS

FIG. 1 is a schematic illustrating the sealants of the invention.

FIG. 2 shows a three-dimensional representation of the sealants of the invention.

FIG. 3 shows an illustration of synthesis of a sealant of the invention comprising identical macromonomers and identical four-armed crosslinkers.

FIG. 4 shows an alternative embodiment wherein the macromonomer is a four-armed polymer containing two functional groups on each arm.

FIG. 5 shows a similar polymer wherein the macromonomer contains three functional groups on each arm.

FIG. 6 sows a similar polymer to that in FIG. 5 except that the crosslinker is a bifunctional polymer.

FIG. 7 shows the rates of drug release and gel degradation from the invention sealants.

FIG. 8 illustrates formation sealant of the invention wherein a 4-arm macromonomer wherein each arm is terminated with linker-azide and HSE groups is crosslinked with a macromonomer wherein each arm is terminated with a cyclooctyne. In this example, the sealant crosslinking involves formation of triazole linkages between macromonomers and tissue adhesion results from reaction of the HSE groups with tissue-surface amines.

FIG. 9 illustrates formation sealant of the invention wherein an 8-arm macromonomer wherein each arm is terminated with linker-succinimidyl carbonate group is crosslinked with a macromonomer wherein each arm is terminated with an amine. In this example, the sealant crosslinking involves formation of carbamate linkages between macromonomers, with the degree of crosslinking being determined by the ratio of the reactive groups on the macromonomers, and tissue adhesion results from reaction of the remaining succinimidyl carbonate groups with tissue-surface amines.

### MODES OF CARRYING OUT THE INVENTION

The invention provides degradable sealants having controlled rates of degradation and optionally having controlled drug release and methods for their preparation and use. These sealants are expected to provide leak-free closures around sutures, surgical anastomoses, surgical implants, and wounds, as well as serve as adhesion barriers, tissue adhesives, and bandages. Degradable sealants have been previously disclosed, although degradation has been achieved by hydrolytic reactions having rates that are poorly controlled and difficult to predict. In the present invention, controlled degradation rates are achieved through the use of beta-elimination linkers, which may be incorporated into the sealant crosslinks, drug attachment connectors, tissue attachment connectors, or combinations thereof.

By “a moiety capable of being cleaved by elimination under physiological conditions” is meant a structure comprising a group —CH—(CH═CH)ₙC—X wherein n is 0 or 1 and X is a leaving group, wherein an elimination reaction to remove the elements of HX can occur at a rate such that the half-life of the reaction is between 1 and 10,000 hours under physiological conditions of pH and temperature. Preferably, the half-life of the reaction is between 1 and 5,000 hours, and more preferably between 1 and 2,500 hours, more preferably between 1 and 1,000 hours under physiological conditions of pH and temperature. By physiological conditions of pH and temperature is meant a pH of between 7 and 8 and a temperature between 30 and 40° C.

It should be noted that when ranges are given in the present application, such as 1-2,500 hours, the intermediate interval numbers should be considered as disclosed as if specifically and explicitly set forth. This avoids the necessity of long list of numbers and applicants clearly intend to include any arbitrary range between the outer boundaries. For example, the range 1-1,000 also includes 1-500 and 2-10.

A “hydrogel” is a three-dimensional, predominantly hydrophilic polymeric network comprising a large quantity of water, formed by chemical or physical crosslinking of natural or synthetic homopolymers, copolymers, or oligomers. The components of the hydrogel that are crosslinked together may be multi-armed polymers. In describing the makeup of the hydrogels of the invention, the components whether single polymers or multi-arm polymers will be referred to as “macromonomers” because they constitute the individual elements in the overall crosslinked structure which is the hydrogel or sealant. Hydrogels may be formed through crosslinking polyethylene glycols (considered to be synonymous with polyethylene oxides), polypropylene glycols, poly(N-vinylpyrrolidone), poly-methacrylates, polyphosphazenes, polylactides, polyacrylamides, polyglycolates, polyethylene imines, agarose, dextran, gelatin, collagen, polylysine, chitosans, alginates, hyaluronans, pectin, and carrageenan. A multi-armed polymer is formed of more than a single chain and typically has an even number of arms, each arm of which may bear one or more functional groups for further reaction. A single-armed polymer is a single chain which may have one or more functional groups at each end. A multi-armed polymer can support more than one or two functional groups at the terminus of each of the arms. Hydrogels may also be environment-sensitive, for example being liquids at low temperature but gelling at 37° C., for example hydrogels formed from poly(N-isopropylacrylamide).

The pore sizes characteristic of the polymer are also variable depending on the concentrations and nature of the reactants used to compose it. A “mesoporous” hydrogel is a hydrogel having pores between approximately 1 nm and approximately 100 nm in diameter. The pores in mesoporous hydrogels are sufficiently large to allow for free diffusion of biological molecules such as proteins. A “macroporous” hydrogel is a hydrogel having pores greater than approximately 100 nm in diameter. A “microporous” hydrogel is a hydrogel having pores less than approximately 1 nm in diameter.

In some embodiments, for example, of the multilayer sealants or hydrogels of the invention, the hydrogel in contact with tissue or intended to be in contact with tissue is a macroporous hydrogel and the upper layer in contact with fluid is the mesoporous hydrogel or a microporous hydrogel. Alternatively, for example, the layer in contact with a tissue may be a mesoporous hydrogel while the layer in contact with the fluid is the microporous hydrogel. Typically, the multilayer hydrogels or sealants will be formed in situ—i.e., the layer in contact with tissue is laid down first, followed by application of the overlaying hydrogel layer. Alternatively, the multilayer hydrogel may be pre-formed and the layer intended for tissue contact be provided with a protein-reactive set of functional groups so as to attach to the tissue itself.

A “biodegradable sealant” is a sealant that loses its structural integrity through the cleavage of component chemical bonds under physiological conditions of pH and temperature. Biodegradation may be enzymatically catalyzed or may be solely dependent upon environmental factors such as pH and temperature. Biodegradation results in formation of fragments of the polymeric network that are sufficiently small to be soluble and thus undergo clearance from the system through the usual physiological pathways. In many embodiments of the present invention, the degradation occurs through an elimination reaction effected by virtue of crosslinkers of formulas (1a) or (2) described above.

“Functional groups” refer to groups of atoms that are reactive towards other functional groups, most preferably under mild conditions compatible with the stability requirements of peptides, proteins, and other biomolecules. Suitable functional groups found in crosslinkers that couple the macromonomers and in the macromonomers themselves of the hydrogel include maleimides, thiols or protected thiols, alcohols, acrylates, acrylamides, amines or protected amines, amino ethers, carboxylic acids or protected carboxylic acids, azides, alkynes including cycloalkynes, 1,3-dienes including cyclopentadienes and furans, cyclooctenes, cyclopropenes, alpha-halocarbonyls, N-hydroxysuccinimide or N-hydroxysulfo-succinimide esters or carbonates, and 1,2,4,5-tetrazines.

Thus, functional groups capable of connecting to the macromonomers are functional groups that react to cognate functional groups of a reactive polymer to form a covalent bond to the macromonomer. These functional groups that are used to assemble the hydrogel are not protein-reactive. Examples of suitable functional groups are illustrated in the embodiments below.

A “protein-reactive” functional group is a group that is capable of reacting directly with a protein in situ. Typically, proteins contain SH groups, COOH groups, and NH₂ groups that are available for reaction with the protein-reactive group. Since the sealant must be operable in situ, reactions that require additional crosslinking such as reaction which require carbodiimide, for example, are not considered “protein-reactive functional groups.” Examples of protein-reactive functional groups include succinimidyl carbonates, succinimidyl ester, maleimides, alpha-halo-carbonyl derivatives and the like. Thus, these protein reactive groups include a hydroxysuccinimide or sulfohydroxysuccinimide ester or carbonate; a substituted phenyl ester or carbonate; a maleimide, vinylsulfone, or vinylsulfonamide; or an alpha-halo ketone, alpha-halo carboxamide, or alpha-halo carboxylate, an aldehyde, or a perfluorohydrocarbyl group.

The “crosslinkers” or “linkers” are compounds comprising at least two functional groups that are capable of forming covalent bonds with one or more reactive macromonomers or other molecules. As used in the present application, “crosslinker” refers to the molecules that join the macromonomers to form the hydrogels and “linkers” refer to simple molecules that couple a protein-reactive group or a drug to the hydrogel. Typically, the reactive macromonomers are soluble, and crosslinking results in formation of an insoluble three-dimensional network or gel. The functional groups of the crosslinking reagent may be identical (homofunctional) or different (heterofunctional). The functional groups of the heterofunctional crosslinking reagent are chosen so as to allow for reaction of one functional group with a cognate group of the reactive macromonomers and reaction of the second functional group with a cognate group of the same or a different macromonomer. The functional groups of a multifunctional crosslinking reagent are chosen so that they are not reactive with themselves, i.e., are not cognates. In typical embodiments of the present invention, the crosslinkers used to construct the hydrogel will be linked via functional groups that are not “protein-reactive groups.” In this instance, there is no possibility that incompletely reacted crosslinkers would provide functional groups that could react with proteins and behave as sealants.

In one aspect the invention provides sealants having controlled rates of degradation and optionally having controlled drug release. The sealants of the invention are hydrogels of crosslinked macromonomers wherein the hydrogel also comprises a plurality of functional groups that promote tissue adherence, i.e., protein-reactive groups. These are linked to the hydrogel optionally through degradable linkers, and wherein the linkers may comprise groups that degrade by a pH-dependent elimination process thus allowing the sealant to be resorbed. In some embodiments, the sealants of the invention further comprise drugs, wherein the drugs are covalently linked to the hydrogel optionally through linkers that degrade by a pH-dependent elimination process thereby releasing the drug.

Thus, some alternative embodiments of the invention are those where a protein-reactive functional group is coupled to a biodegradable hydrogel through linkers that undergo elimination reactions to control release of the moiety containing the protein-reactive functional group when the sealant has been bound to protein, but wherein the remainder of the polymer is degradable by conventional methods.

In some embodiments, the protein-reactive functional group containing moiety is coupled to the hydrogel not so as to be releasable under physiological conditions, but the hydrogel itself is biodegradable by virtue of crosslinkers that undergo elimination reactions under physiological conditions. In other embodiments, both the moiety containing the protein-reactive functional group and the hydrogel itself are crosslinked through groups that undergo biodegradation through an elimination reaction. In those embodiments wherein a drug is coupled to the hydrogel, unless the hydrogel is completely biodegradable, the drug should be releasably linked to the hydrogel, optionally, though not necessarily, through a linker that undergoes elimination to release the drug.

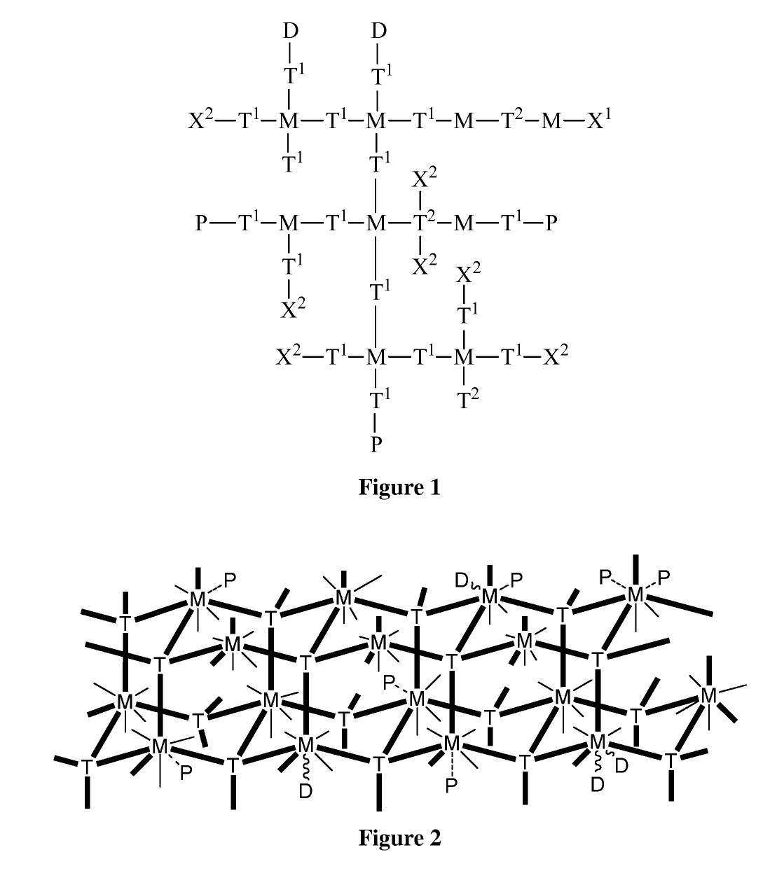

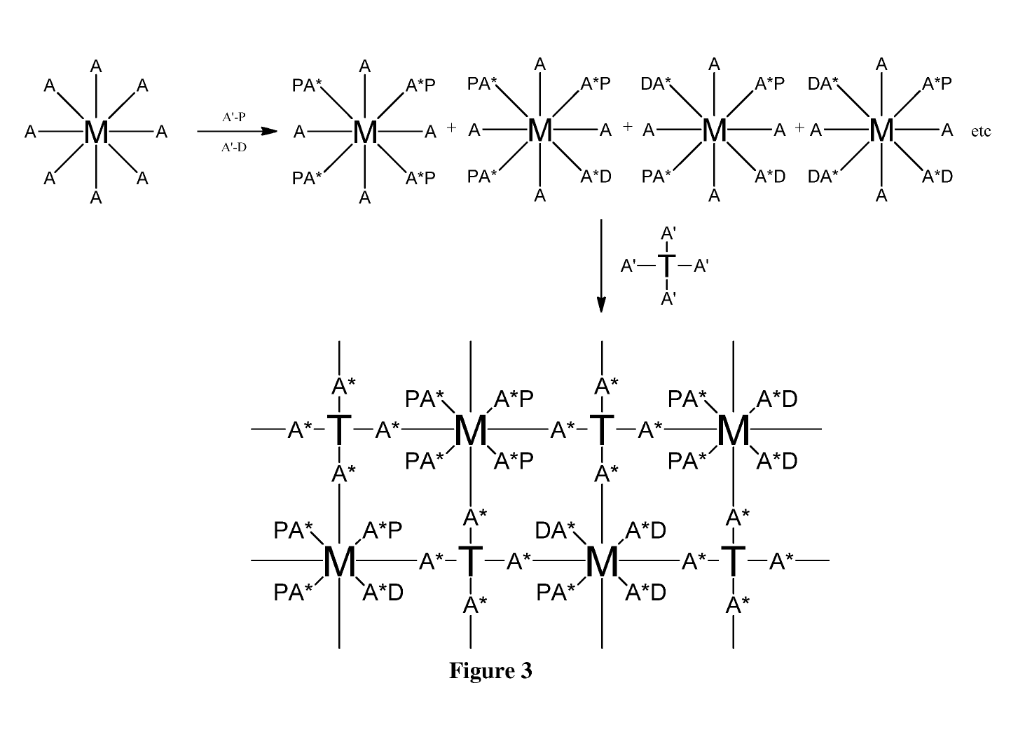

An illustrative embodiment of the sealants of the invention is shown in FIG. 1. The illustration is, of course, only a schematic, and not intended to represent even one particular embodiment of a specific chemical makeup, but rather to illustrate the nature of the variable features of the sealants included within the invention. As shown in FIG. 1, the gel is made up of eight polymers symbolized by “M” that are crosslinked through various crosslinkers indicated as T¹ and T². Crosslinkers T¹ are bifunctional and in some embodiments, a plurality of the T¹ crosslinkers are of formula (1a). The crosslinkers designated T² are multi-armed crosslinkers and in some embodiments are of formula (2). Not all of the T¹ and T² crosslinkers need be the same. Not all of the functional groups of all of the crosslinkers are reacted with other substituents or with the macromonomers of the gel. In these cases, the unreacted functional group is shown as X². For some of the multi-armed crosslinkers T², not all of the arms need have functional groups as shown, for example, in the lower right-hand corner of the figure. While the illustration in FIG. 1 shows the possibility for two types of crosslinkers in a single hydrogel, it is more frequently convenient to prepare hydrogels having only one type of crosslinker. Additional FIGS. 3-6 illustrate these possibilities.

Some of the crosslinkers or macromonomers are bound to tissue adherence functional groups—i.e., protein-reactive groups designated “P” in the figure. These are coupled to the remainder of the gel typically by bifunctional linkers. If the biodegradable hydrogel does not contain a plurality of crosslinkers that degrade through elimination as described above, the linker coupled to the protein-reactive group should be capable of degradation through the elimination reaction described. In the illustration shown in FIG. 1, X² is not a protein-reactive functional group; the protein-reactive functional group is supplied by P. Of course, in some embodiments the protein-reactive groups are coupled to the hydrogel through linkers cleavable by elimination and the hydrogel itself is crosslinked using such linkers.

It is optional, but the matrix may also include a drug, symbolized by “D” which itself may be coupled to the gel through a linker which is optionally and preferably biodegradable, more preferably through cleavage by an elimination reaction. Preferably, the bifunctional linkers shown as T¹ in the figure that couple D to the remainder of the polymer are biodegradable through an elimination reaction—i.e., are of formula (1b). If at least the linker coupling P or a majority of the linkers coupling the polymers of the hydrogel are linked by crosslinkers that degrade by the elimination reaction, the crosslinker shown as T¹ binding D to the remainder of the hydrogel may be biodegradable by other mechanisms.

Of course, more than one type of drug can be included in the structure and more than one type of protein-reactive group may be included. It should be noted as well that some of the crosslinkers, labeled T², may form part of the hydrogel itself by virtue of their nature—i.e., the 4 arms shown as T² may be coupled to a polymeric center; i.e., T² may be of formula (2).

In FIG. 1, the functional groups shown as X² need not all be identical either. The linkage between the polymer shown as M and the crosslinkers shown as T are through coupling of a functional group on M, which can be designated X¹ with a cognate functional group X² on the crosslinker. The result of binding effected by X¹ will be a residue of the reaction of these two groups.

FIG. 2 shows a more detailed three-dimensional rendering of the schematic shown in FIG. 1. In this embodiment, M is an 8-arm polymer; T is a 4-arm crosslinker; and q is 4. Each M and T are connected through a crosslinker (heavy lines). The remaining arms on M (light lines) may be connected to groups P and D through linkers (dashed lines) and (squiggly lines), respectively. Not every M need connect to a P and/or D; some M may connect with more than one P and/or D; thus, m and n may be non-integral numbers. While a regular polymer structure is given for purposes of illustration, q may also be non-integral leading to an irregular polymer structure.

The complicated-looking hydrogel structures of the invention in FIGS. 1 and 2 are summarized with the perhaps oversimplified formula

(MTₓ)_{y} (3)

where M is a multivalent polymer (i.e., two or more reactive functional groups),

T is a crosslinker,

x is an integer of 2-20 or 2-40, and

y is an integer that results in the hydrogel.

The hydrogel itself can then be coupled to protein-reactive groups, P, and optionally to drug, D.

As is evident from FIGS. 1 and 2, there must be a multiplicity of macromonomers, some of which are multi-armed, but some of which may contain only two functional groups; the various macromonomers are linked together by the same or different crosslinkers and some of the crosslinkers will be left over still bearing a functional group, which, however, is not protein-reactive unless already bound to protein. This, however, is perhaps an oversimplification as the remaining functional groups can be capped by protective groups or a small number of monofunctional polymers could be included behaving as caps.

FIGS. 3-6 show illustrative alternative embodiments of the sealants of the invention.

FIG. 3 illustrates one embodiment of sealants of the invention prepared from crosslinking of an 8-arm M wherein each arm is terminated with reactive group A with a 4-arm T wherein each arm is terminated with cognate reactive group A′. Four of the A groups of M are reacted with a mixture of A′-P and A′-D to attach tissue adhesive groups P and drugs D to M via residue A*. This provides a mixture of derivatized M comprising random arrangements of P, D, and residual A groups, with the mixture determined by reaction stoichiometry. The mixture is crosslinked with the 4-arm T to provide the sealant.

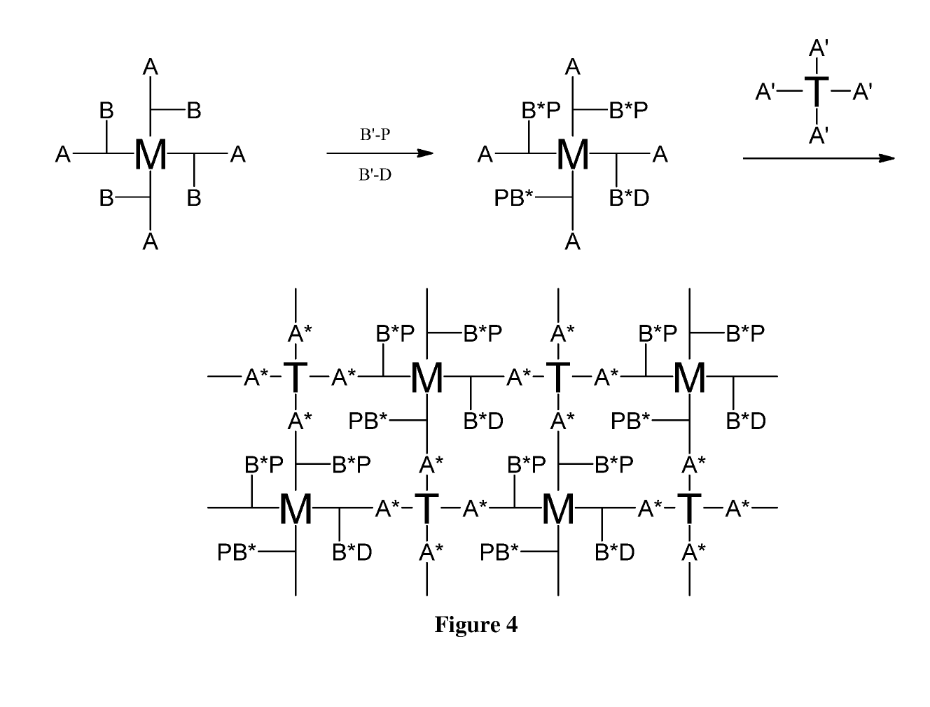

FIG. 4 illustrates one embodiment of sealants of the invention prepared from crosslinking of M wherein each arm of M is terminated by two reactive functional groups X=A or B with a tetrafunctional crosslinker T. X=A is reacted with cognate function group A′ to crosslink the sealant via remnant A*. X=B is reacted with cognate function group B′ to link the tissue-adhesive group P via remnant B*. Some X=B optionally may be reacted with cognate function group B′ to link a drug D via remnant B*. This method provides sealants having more controlled stoichiometries.

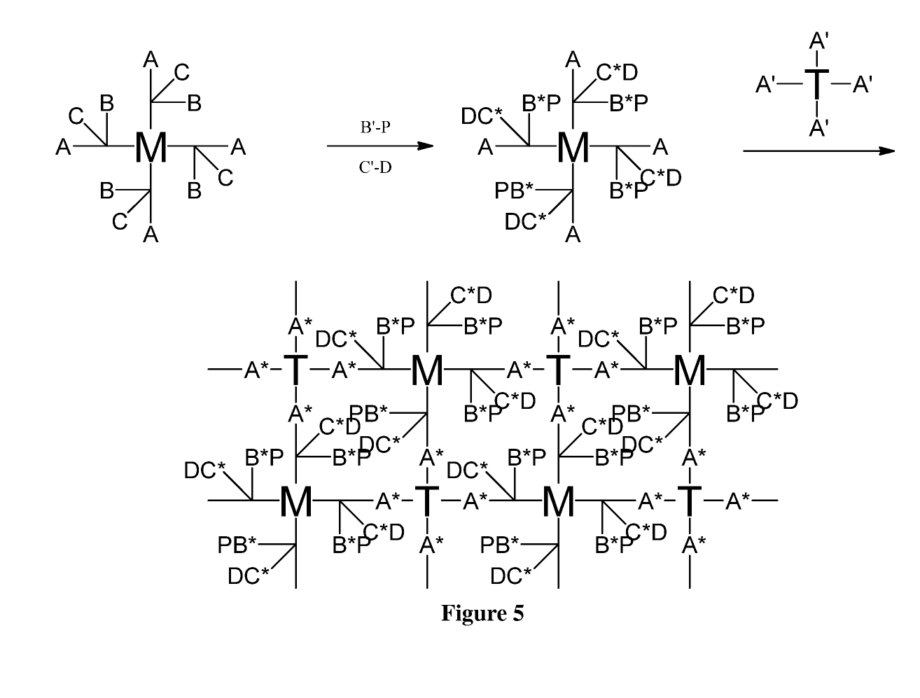

FIG. 5 illustrates one embodiment of sealants of the invention prepared from crosslinking of M wherein each arm of M is terminated by three reactive functional groups X=A, B, or C with a tetrafunctional crosslinker T. X=A is reacted with cognate function group A′ to crosslink the sealant via remnant A*. X=B is reacted with cognate function group B′ to link the tissue-adhesive group P via remnant B*. X=C is reacted with cognate function group C′ to link a drug D via remnant C*. This method provides sealants having more controlled stoichiometries.

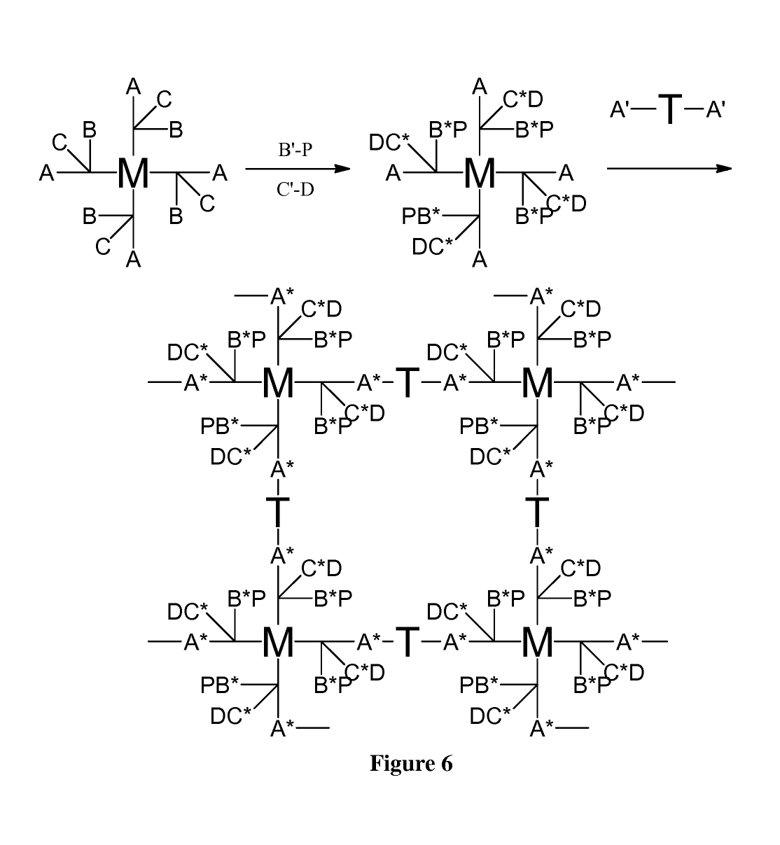

FIG. 6 illustrates one embodiment of sealants of the invention prepared from crosslinking of M wherein each arm of M is terminated by three reactive functional groups X=A, B, or C with a bifunctional crosslinker T. X=A is reacted with cognate function group A′ to crosslink the sealant via remnant A*. X=B is reacted with cognate function group B′ to link the tissue-adhesive group P via remnant B*. X=C is reacted with cognate function group C′ to link a drug D via remnant C*. This method provides sealants having more controlled stoichiometries.

### Embodiments of M

In each embodiment M may be homopolymeric or copolymeric poly(ethylene glycol)s or poly(ethylene oxide)s (PEG or PEO), polypropylene glycols (PPG), poly(N-vinyl-pyrrolidone), polymethacrylates, polyphosphazenes, polylactides, polyacrylamides, polyglycolates, poly(ethyleneimine)s, agaroses, dextrans, gelatins, collagens, polylysines, chitosans, alginates, hyaluronans, pectins, or carrageenans that either comprise suitable reactive functionalities in their native state or have been derivatized so as to comprise suitable reactive functionalities X¹. Native polymers that do not comprise an effective multiplicity of reactive groups can be transformed by reaction with reagents that introduce an effective multiplicity of reactive groups prior to formation of the hydrogel using methods well known in the art. The macromonomer may comprise multivalent branched structures. Examples include multivalent star-shaped polymers, for example those based on pentaerythritol, and comb-shaped polymers, for example those based on derivitization of hexaglycerin or tripentaerythritol (see core structures below). The number of monomer units comprising the macromonomer can be 10-1,000 or intermediate values such as 20, 50, 100, etc. This listing is intended to include all intermediate integers between 10 and 1,000, as most commercially available polymers are mixtures comprising a distribution of different monomer numbers. M is typically of molecular weight between 1,000 and 150,000; preferably between 1,000 and 70,000. In some embodiments, M is a protein, for example an albumin or fibrin, having a multiplicity of reactive amine groups from surface lysine residues. It may be necessary to provide an adaptor to supply a functional group cognate to a functional group that is not protein-reactive in this case. For instance, a heterofunctional linker wherein one group reacts with amines, sulfhydryl and carboxy and a second group such as an azide can be used.

M may also comprise multiple arms wherein each arm is terminated with at least two functional groups X¹, wherein each X¹ on an arm may be the same or different. In one embodiment, each arm is terminated with two functional groups X¹. As examples, one X¹ may be an azide and the other an aldehyde (if already bound to protein), or one X¹ may be a cyclooctyne while the other is a cyclopropene, or one X¹ may be a highly reactive cyclooctyne such as DBCO while the other is a relatively unreactive cyclooctyne such as MOFO. In the first example, the azide can be used to couple or crosslink using 1,3-dipolarcycloaddition reactions. In the second example, the cyclooctyne can be used to couple or crosslink with an azide using 1,3-dipolarcycloaddition reactions while the cyclopropene may be used to couple or crosslink to a tetrazine using a Diels-Alder reaction. In the third example, the two different cyclooctynes can be used to couple to two different azides based on the differential rates of reaction. Other such combinations will be apparent. In another embodiment, each arm of M is terminated with three functional groups X¹, allowing for control over sealant crosslinking, protein attachment group linking, and drug linking. For example, each arm of M may be terminated with a cyclooctyne, a cyclopropene, and an aldehyde group.

### Embodiments of X

In some embodiments, the functional groups X¹ and X³ employed to form the matrix itself will not include those wherein one of the cognates is a group found in protein so that adherence to the tissue would be effected by any leftover reactive groups. Thus, the cognate pairs in the formation of the hydrogels will be those that are not interactive with any carboxyl, amino, or sulfhydryl groups. Examples of these groups include those wherein one of X¹ and X² has a terminal acetylene, 1,1,1-trifluoro-propyne, or cyclooctyne moiety and the other is a group capable of undergoing a 1,3-dipolar cycloaddition, such as N₃ resulting in formation of a triazole linkage, or a nitrone resulting in isoxazoline formation (see, for example, Ning, et al., “Protein Modification by Strain-promoted Alkyne-Nitrone Cycloaddition,” Ang. Chem. Int. Ed.

(2010) 49:3065-3068). Examples of moieties comprising cyclooctynes include dibenzocyclooctynes (DBCO, DIBO, BARAC, DIBAC), fluorocyclooctynes (MOFO, DIFO, DIFO2, DIFO3), strained bicyclic cyclooctynes such as bicyclononynes (BCN), and others known in the art (see, for example, Marjoke F. Debets, et al., “Bioconjugation with Strained Alkenes and Alkynes,” Accounts of Chemical Research (2011) 44:805-815, incorporated herein by reference). The 1,1,1-trifluoropropyne may be generated in situ from the corresponding Diels-Alder adduct with furan. When one group is a terminal acetylene, the reaction is catalyzed by addition of a metal ion such as copper.

Another example is that wherein one of X¹ and X³ comprises a 1,2,4,5-tetrazine, and the other is a trans-cyclooctene, norbornene, or cyclopropene (see for example, Karver, et al., “Bioorthogonal Reaction Pairs Enable Simultaneous, Selective, Multi-target Imaging,” Ang. Chem. Int. Ed. (2012) 51:920-922; Yang, et al., “Live-Cell Imaging of Cyclopropene Tags with Fluorogenic Tetrazine Cycloadditions,” Ang. Chem. Int. Ed. (2012) 51:7476-7479; and Devaraj, “Advancing Tetrazine Bioorthogonal Reactions through the Development of New Synthetic Tools,” Synlett (2012) 23:2147-2152, each of which is incorporated herein by reference). These groups react via Diels-Alder reaction to provide a stable pyridazine linkage.

However, with respect to any cleavable linker that provides a protein-reactive group, X² may be such a group. For example, if X² comprises a hydroxysuccinimide ester or carbonate moiety it can bind to a thiol or an amine, resulting in formation of a thioester, thiocarbonate, amide, or carbamate linkage, respectively. If X² comprises a maleimide, vinylsulfone, vinylsulfonamide, acrylate, or acrylamide, it can bind a thiol, resulting in formation of a thioether linkage.

If X² comprises an aldehyde it can bind an amine, resulting in formation of an imine or it can bind an NH₂CHCH₂SH moiety of a cysteine, resulting in formation of an amide linkage via native chemical ligation or a pseudoproline linkage via pseudoproline peptide ligation (Hu, et al., “Hydrogels cross-linked by native chemical ligation,” Biomacromolecules (2009) 10:2194-2200; and Wathier, et al., “Hydrogels formed by multiple peptide ligation reactions to fasten corneal transplants,” Bioconjugate Chem. (2006) 17:873-876).

### Embodiments of T

Bifunctional crosslinkers that undergo 0 elimination have been previously disclosed, for example in Santi, et al., “Predictable and Tunable Half-life Extension of Therapeutic Agents by Controlled Chemical Release from Macromolecular Conjugates,” Proc. Natl. Acad. Sci USA (2012) 109:6211-6216; PCT publications WO2009/158668 and WO2011/140393; and PCT application US/2012/54293, which are hereby incorporated by reference and are of the formula

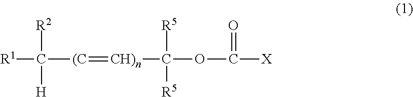

wherein

n is 0 or 1;

X is a group that binds with the components of the hydrogel or other moiety;

at least one or both R¹ and R² is independently CN; NO₂;
optionally substituted aryl;
optionally substituted heteroaryl;
optionally substituted alkenyl;
optionally substituted alkynyl;
COR³ or SOR³ or SO₂R³ wherein
   R³ is H or optionally substituted alkyl;
   aryl or arylalkyl, each optionally substituted;
   heteroaryl or heteroarylalkyl, each optionally substituted; or
   OR⁹ or NR⁹₂ wherein each R is independently H or optionally substituted alkyl, or both R⁹ groups taken together with the nitrogen to which they are attached form a heterocyclic ring;
SR⁴ wherein
   R⁴ is optionally substituted alkyl;
   aryl or arylalkyl, each optionally substituted; or
   heteroaryl or heteroarylalkyl, each optionally substituted;

wherein R¹ and R² may be joined to form a 3-8 membered ring; and

wherein one and only one of R¹ and R² may be H or may be alkyl, arylalkyl or heteroarylalkyl, each optionally substituted; and

each R⁵ is independently H or is alkyl, alkenylalkyl, alkynylalkyl, (CH₂CH₂O)ₚ wherein p=1-1000, aryl, arylalkyl, heteroaryl or heteroarylalkyl, each optionally substituted; and

wherein at least one of R¹, R², and R⁵ is substituted with X, wherein X is a functional group for binding to X¹. In some embodiments, X does not bind directly to protein.

The crosslinking reagents for the hydrogel also include multivalent compounds of the formula

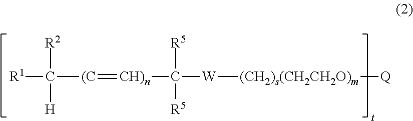

wherein one of R¹, R² and R⁵ in at least two of the t moieties shown within the bracket comprises the functional group X³ wherein in some embodiments X³ does not react with protein and R¹, R² and R⁵ are otherwise defined above and wherein

n is 0 or 1;

m is 0-1,000;

s is 0-2;

t is 2, 4, 8, 16 or 32,

W is O(C═O)O, O(C═O)NH, O(C═O), S,

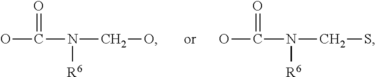

wherein R⁶ is as defined above; and

Q is a core group having a valency=t.

The core group Q is a group of valency =t which connects the multiple arms of the crosslinking reagent. Typical examples of Q include an ethylene core CH₂CH₂ (t=2), pentaerythritol core C(CH₂)₄ (t=4); a hexaglycerin core (t=8); and a tripentaerythritol core (t=8).

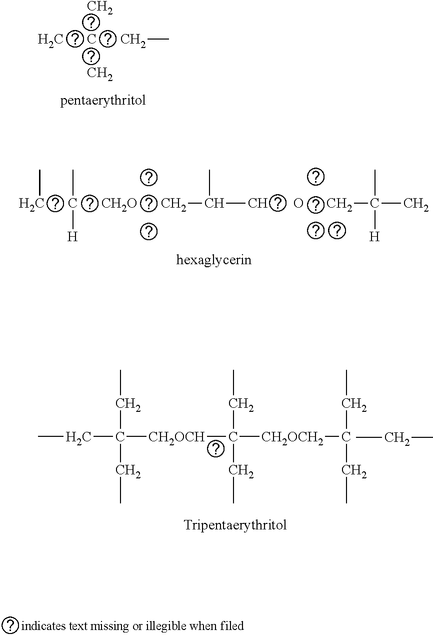

Compounds of formula (2) may be prepared by the reaction of a multi-arm polyethylene glycol with a suitable reagent as disclosed in PCT application US2012/54278, which is incorporated herein by reference. A variety of multi-arm polyethylene glycols are commercially available, for example from NOF Corporation and JenKem Technologies.

The linkers of formulas (1), (1a), (1b) and (2) degrade through a non-hydrolytic elimination mechanism, with the rates of release being controlled primarily by the groups R¹ and R², and to a lesser extent R⁵. The properties of R¹ and R² may be modulated by the optional addition of electron-donating or electron-withdrawing substituents. By the term “electron-donating group” is meant a substituent resulting in a decrease in the acidity of the R¹R²CH; electron-donating groups are typically associated with negative Hammett σ or Taft σ* constants and are well-known in the art of physical organic chemistry. (Hammett constants refer to aryl/heteroaryl substituents, Taft constants refer to substituents on non-aromatic moieties.) Examples of suitable electron-donating substituents include but are not limited to lower alkyl, lower alkoxy, lower alkylthio, amino, alkylamino, dialkylamino, and silyl. Similarly, by “electron-withdrawing group” is meant a substituent resulting in an increase in the acidity of the R¹R²CH group; electron-withdrawing groups are typically associated with positive Hammett σ or Taft σ* constants and are well-known in the art of physical organic chemistry. Examples of suitable electron-withdrawing substituents include but are not limited to halogen, difluoromethyl, trifluoromethyl, nitro, cyano, C(=O)—R^{X}, wherein R^{X} is H, lower alkyl, lower alkoxy, or amino, or S(O)ₘR^{Y}, wherein m=1-2 and R^{Y} is lower alkyl, aryl, or heteroaryl. As is well-known in the art, the electronic influence of a substituent group may depend upon the position of the substituent. For example, an alkoxy substituent on the ortho- or para-position of an aryl ring is electron-donating, and is characterized by a negative Hammett σ constant, while an alkoxy substituent on the meta-position of an aryl ring is electron-withdrawing and is characterized by a positive Hammett σ constant. A table of Hammett σ and Taft σ* constants values is given below.

| Substituent | σ(meta) | σ(para) | σ* |
|---|---|---|---|
| H | 0.00 | 0.00 | 0.49 |
| CH₃ | −0.07 | −0.17 | 0 |
| C₂H₅ | −0.07 | −0.15 | −0.10 |
| n-C₃H₇ | −0.07 | −0.13 | −0.115 |
| i-C₃H₇ | −0.07 | −0.15 | −0.19 |
| n-C₄H₉ | −0.08 | −0.16 | −0.13 |
| t-C₄H₉ | −0.10 | −0.20 | −0.30 |
| H₂C═CH | 0.05 | −0.02 | |
| C₆H₅ | 0.06 | −0.01 | 0.60 |
| CH₂C1 | 0.11 | 0.12 | 1.05 |
| CF₃ | 0.43 | 0.54 | |
| CN | 0.56 | 0.66 | |
| CHO | 0.35 | 0.42 | |
| COCH₃ | 0.38 | 0.50 | |
| CO₂H | 0.37 | 0.45 | |
| Si(CH₃)₃ | −0.04 | −0.07 | |
| F | 0.34 | 0.06 | |
| Cl | 0.37 | 0.23 | |
| Br | 0.39 | 0.23 | |
| I | 0.35 | 0.18 | |
| OH | 0.12 | −0.37 | |
| OCH₃ | 0.12 | −0.27 | |
| OCH₂CH₃ | 0.10 | −0.24 | |
| SH | 0.25 | 0.15 | |
| SCH₃ | 0.15 | 0.00 | |
| NO₂ | 0.71 | 0.78 | |
| NO | 0.62 | 0.91 | |
| NH₂ | −0.16 | −0.66 | |
| NHCHO | 0.19 | 0.00 | |
| NHCOCH₃ | 0.07 | −0.15 | |
| N(CH₃)₂ | −0.15 | −0.83 | |
| N(CH₃)⁺ | 0.88 | 0.82 | |
| CCl₃ | | | 2.65 |
| CO₂CH₃ | | | 2.00 |
| CH₂NO₂ | | | 1.40 |
| CH₂CF₃ | | | 0.92 |
| CH₂OCH₃ | | | 0.52 |
| CH₂Ph | | | 0.26 |

Alkyl R⁵ groups slow the elimination reaction slightly relative to aryl R⁵ groups, and so may also be used to tune the rates of elimination and degradation.

### Embodiments of P

Tissue adherence of the sealant is enhanced by reaction of a protein-reactive functional group P with the tissue matrix. P is embodied in one X² in formula (1). For example, reaction of P with complementary functional groups on tissue proteins may provide adherence of the sealant with the tissue. Most commonly, the available protein functional groups will be amines, such that P is a group reactive with amines, for example N-hydroxysuccinimide ester or carbonate. Other groups reactive with amines may be used, including 1,3-diketones, aldehydes, and ketones. P may also be a group reactive towards protein thiols, including maleimide, vinylsulfone, vinylsulfonamide, disulfide, haloacetyl, haloacetamide, acrylate, and acrylamide.

### Embodiments of D

A variety of drugs D may optionally be attached to the sealant in order to enhance wound healing. In particular, antibiotics including antibacterials, antifungals, and antivirals; hormones including steroids such as triamcinolone, triamcinolone acetonide, dexamethasone, betamethasone, prednisone, prednisolone, rimexolone, and derivatives thereof; immunosuppressants including FK506 and rapamycin; cytostatic agents including 5-fluorouracil and tubulin inhibitors such as paclitaxel, docetaxel, vincristine, and epothilones; peptides and proteins including growth factors, coagulating agents, and antibodies; and nucleic acids including aptamers and siRNA may be used. A variety of growth factors have been found to play a role in wound healing and thus may be used in the invention, including platelet-derived growth factors (PDGF), bone morphogenetic factors such as BMP-2 and BMP-7, epidermal growth factors (EGF), fibroblast growth factors such as bFGF and FGF-2, transforming growth factors like TGF-β1, vascular endothelial growth factors (VEGF), hepatocyte growth factors (HGF), keratinocyte growth factors (KGF), and insulin-like growth factors like IGF-1. The preparation of linker-drugs X²-L₂-D is detailed in PCT publications WO2009/158668 and WO/2011/140393, which are hereby incorporated by reference.

### Embodiments of A

In some embodiments, an adapter unit A may be present to introduce multiple functionality at the end of each arm of a reactive polymer M-(T)_{q}. Unit A comprises a functional group X⁵ that is reactive with functional groups terminating the arms of reactive polymer M together with at least two functional groups, which may be the same or different:

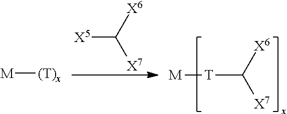

Typical examples of suitable adapters A include derivatives of lysine, aspartic acid, or glutamic acid. If these are used as crosslinkers, further conversion of amino, carboxyl or sulfhydryl groups to cognates of groups that are not protein-reactive is needed.

### Preparation Methods

In other aspects the invention provides methods for the preparation of the sealants of the invention. The sealant forming reactions may be performed in a variety of suitable solvents, for example water, alcohols, acetonitrile, or tetrahydrofuran, but are preferably performed in aqueous medium optionally in the presence of small amounts of organic cosolvents. Formation of the sealants may be performed in a stepwise or a concerted fashion. Thus, in one embodiment of the invention, a first solution comprising the hydrogel is mixed with a second solution comprising the moiety comprising the protein-reactive group, preferably of formula (1). The compound of formula (1) containing drug D may also be included. Any order of reaction may be used.

The molar ratios of the components in the polymerization mix may be adjusted to control the physical properties of the sealant, the drug content, and the attachment of the sealant to tissue. For example, the physical properties of the sealant may be controlled through appropriate selection of hydrogel with the degree of its crosslinking, and the ratio of the moiety comprising the protein-reactive functional group.

The nature of the sealant as related to the nature of the hydrogel itself is controlled by the level of crosslinking. This is determined by the value of q in the crosslinker of formula (2). Generally speaking, as q increases the sealant becomes stiffer and more durable, with the upper bound of q being determined by the difference in the number of reactive groups on M and the sum of the number of drugs D and tissue adherent groups P concurrently attached to M. It is not necessary for all reactive groups on M to be bonded to T, D, and P groups, and given the polymeric nature of the sealants of the invention it is not necessary for every M unit in the sealant to be bonded to a P and/or D group, such that taken on average across the bulk polymeric sealant, the number n of D groups and m of P groups per M unit may be non-integer ratios. In some embodiments, the number of D groups is 0.

The polymer content of the final water-swollen sealants may be between 1 and 50% w/v. In some embodiments, the polymer content is between 1 and 25% w/v. In some embodiments, the polymer content is between 1 and 10% w/v.

Some illustrative methods are shown in FIGS. 3-6. In FIG. 3, a protein-reactive functional group coupled to the linker of formula (1) and a drug coupled to a linker of Formula (1b) are first reacted with a macromonomer which macromonomer, now derivatized partly to drug and protein-reactive functional group, is crosslinked with a crosslinker of formula (2).

In FIG. 4, a four-armed macromonomer containing two functional groups on each arm is first reacted with protein-reactive functional groups and drugs each coupled to a linker to provide an intermediate which contains an alternative functional group for binding to A* indicated on each of four arms of crosslinker of formula (2). In FIG. 5, a similar sequence of steps is conducted, but with three functional groups attached to each arm of the macromonomer.

FIG. 6, again, the linkers containing the drug and protein-reactive group are first reacted to functional groups on arms of the macromonomer, M, which is subsequently reacted with crosslinker of formula (1a).

The components of the polymerization mixture may be supplied as dried solids or as suspensions or solutions, for example as aqueous solutions optionally in the presence of buffers, antioxidants, or pharmaceutically acceptable excipients. When supplied as dry solids, the components may also contain excipients in dry form and may be reconstituted with sterile water prior to use or may be dissolved directly into a solution comprising other components of the sealant mixture. In some embodiments, the reactivity of one or more component is modulated by control of the pH of the solution. Pharmaceutically acceptable dyes may be added to enhance visualization of the sealant.

### Method of Use

The sealant is applied to the site requiring sealing and allowed to set. Application may be as a bulk liquid, for example by extrusion from a syringe onto the wound, or by aerosol, for example using a spray device. Two solutions may be premixed or mixed during application using a multi-channel device, for example a multi-barrel syringe wherein each barrel contains a component of the polymerization mixture and the reactive components are mixed upon extrusion into the syringe tip or needle. Devices for application of surgical sealants have been disclosed, for example in U.S. Pat. No. 8,343,183 (issued 1 Jan. 2013) and U.S. Pat. No. 8,262,608 (issued 11 Sep. 2012).

In an alternate embodiment of the invention, the sealant may be preformed and then used as a surgical implant. The sealant may be formed into specific shapes through molding or cutting, and then applied to the wound in the polymeric form.

### Multilayer Sealants

In another aspect the invention provides multi-layer hydrogels and sealants. The multi-layer hydrogels or sealants of the invention are degradable hydrogels comprising at least two layers, wherein each layer is a hydrogel formed from polymers, some of which are multi-armed polymers coupled through biodegradable linkages and wherein the layers of the multilayer hydrogel or sealant are coupled covalently to each other.

In various embodiments of the invention, the successive layers of the multi-layer hydrogel have different degradation rates, or successive layers have different elastic moduli or other physical characteristics such as polymer molecular weight or wt % polymer, or successive layers comprise different releasable drugs D, or successive layers comprise one or more drugs D attached via different releasable linkers or one layer comprises a releasable drug D while the other does not. In one specific embodiment of the invention, one layer of the sealant comprises a tissue-reactive group P while the other comprises a peptide or protein drug D.

In some embodiments, the coupling is through functional groups themselves used to form the individual layers, and which are unreacted in the hydrogel formation of each layer. In some embodiments, at least one of the layers contains a plurality of protein-reactive functional groups so that the multilayer hydrogel behaves as a sealant. In some embodiments, one of the layers comprises a drug. The plurality of protein-reactive functional groups is coupled to at least a first layer through a biodegradable linkage and, in some embodiments, this linkage is degradable through an elimination reaction. The hydrogels themselves are typically biodegradable and the crosslinkers conferring capability for biodegradation may respond to enzymatic or other types of cleavage and in some embodiments are biodegradable by virtue of crosslinkers that are cleavable by an elimination reaction. Thus, the hydrogels and sealants described in the cited PCT application PCT/US2012/54278 and the sealants described in the present application may form the first and second layers. Typically, a first layer intended to be adjacent to tissue will comprise a sealant (i.e., contains said plurality of protein-reactive functional groups) and the second layer, intended to overlay the first layer and in contact with a biological fluid ordinarily in contact with the tissue would also comprise a hydrogel as above described. Such an arrangement is especially useful for drug delivery; the first layer would thus contain drug in a form that can be released into the tissue and the second layer serves a protective function with respect to the drug. Thus, the multilayer hydrogel or sealants of the invention in one embodiment comprise at least a first layer having a first pore size and a second layer overlaying the first layer and coupled thereto said second layer having a different pore size from the first layer.

Where the second layer is intended to shield the first layer which may be adjacent a tissue, the pore size of the second layer will be smaller than that of the first. For example, the first layer may have a pore size with an average diameter of >100 nm and the second layer has a pore size with an average diameter of 1-100 nm. Alternatively, the first layer may have a pore size with an average diameter of 1-100 nm and the second layer a pore size of <1 nm average diameter. In some embodiments, the first layer has a pore size of average diameter of more than 100 nm and the second layer has a pore size of an average diameter of 1-100 nm. In multilayer sealants or hydrogels with more than two layers, varying pore sizes may also be present in each layer.

The multilayer hydrogels and sealants of the invention may be synthesized ex vivo. If so, they may be implanted as such in a subject for sealing tissue or drug delivery or other medical purposes. Alternatively, the multilayer hydrogels or sealants may be formed in situ laying a first layer over the tissue and a second layer atop the first and so on.

Various coupling techniques may be employed; essentially the layers are coupled through cognate functional groups. The cognate functional groups are typically selected from those set forth above for a formation of hydrogels and binding of drugs or protein-reactive groups thereto. The functional groups may constitute those useful in formation of the hydrogels that have been left unreactive by hydrogel formation. For this purpose, in forming at least a bilayer, the cognate functional groups need not exclude protein-reactive groups.

As noted above, in one particularly useful embodiment, a first layer with larger pores than the second will comprise at least one drug coupled thereto by a biodegradable linkage and in some cases by a linker of formula (1b).

The bilayers of the invention are typically biodegradable and preferably biodegradable by virtue of a plurality of crosslinking molecules that are cleavable by an elimination reaction.

All references cited herein are hereby incorporated by reference in their entirety. The invention is further illustrated but not limited by the following examples.

### Preparation A

### Preparation of Azidoalcohols

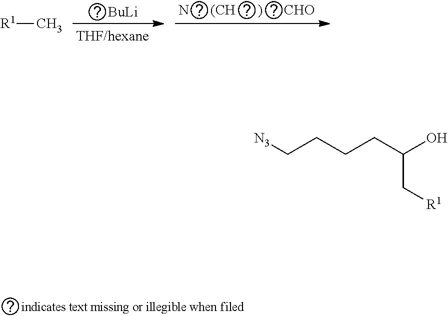

A 1.6 M solution of n-butyllithium (3.1 mL, 5.0 mMol) in hexane was added dropwise to a stirred solution of R¹CH₃ (5.0 mMol) in anhydrous tetrahydrofuran (THF) (15 mL) cooled to −78° C. After addition, the cooling bath was removed and the mixture was allowed to warm slowly to 0° C. over approximately 30 min. The mixture was then cooled back to −78° C., and 6-azidohexanal (5.5 mMol) was added. After stirring for 15 minutes, the cooling bath was removed and the mixture was allowed to warm. At the point where the mixture became clear, 5 mL of saturated aq. NH₄Cl was added and the mixture was allowed to continue warming to ambient temperature. The mixture was diluted with ethyl acetate and washed successively with water and brine, and then dried over MgSO₄, filtered, and evaporated to provide the crude product as an oil. Chromatography on silica gel using a gradient of ethyl acetate in hexane provided the purified products. Compounds prepared according to this method include:

1-(4-(trifluoromethyl)phenylsulfonyl)-7-azido-2-heptanol (R¹=4-(trifluoro-methyl)phenyl-SO₂): from 4-(trifluoromethyl)phenyl methyl sulfone (1.73 g, 94%): ¹H-NMR (400 MHz, CDCl₃): δ8.08 (2H, d, J=8.4-Hz), 7.87 (2H, d, J=8.4-Hz), 4.21 (1H, m), 3.25 (2H, t, J=6.8-Hz), 3.28 (1H, dd, J=8.8, 14.4-Hz), 3.20 (1H, dd, J=2.0, 14.4-Hz), 3.12 (1H, d, J=2.8-Hz), 1.58 (2H, m), 1.5˜1.3 (6H, m);

1-(4-chlorophenylsulfonyl)-7-azido-2-heptanol (R¹=4-chlorophenyl-SO₂): from 4-chlorophenyl methyl sulfone; colorless oil (1.49 g, 90% yield): ¹H-NMR (400 MHz, d₆-DMSO): δ7.90 (2H, d, J=8.8-Hz), 7.70 (2H, d, J=8.8-Hz), 4.83 (1H, d, J=6-Hz), 3.86 (1H, m), 3.39 (2H, m), 3.29 (2H, t, J=6.8-Hz), 1.2˜1.5 (8H, m);

1-(phenylsulfonyl)-7-azido-2-heptanol (R¹=phenyl-SO₂): from phenyl methyl sulfone; pale yellow oil (1.25 g, 85%): ¹H-NMR (400 MHz, d₆-DMSO): δ7.89 (2H, m), 7.72 (1H, m), 7.63 (2H, m), 4.84 (1H, d J=6-Hz), 3.86 (1H, m), 3.33 (2H, m), 3.28 (2H, t, J=6.8-Hz), 1.47 (2H, m), 1.2˜1.4 (6H, m);

1-(4-methylphenylsulfonyl)-7-azido-2-heptanol (R¹=4-methylphenyl-SO₂): from 4-(methylsulfonyl)toluene; colorless oil (1.39 g, 85% yield): ¹H-NMR (400 MHz, d₆-DMSO): δ7.76 (2H, d, J=6.4-Hz), 7.43 (2H, d, J=6.4-Hz), 4.82 (1H, d, J=6-Hz), 3.85 (1H, m), 3.31 (2H, m), 3.28 (2H, t, J=6.8-Hz), 2.41 (3H, s), 1.4˜1.5 (2H, m), 1.2˜1.4 (6H, m);

1-(4-methoxyphenylsulfonyl)-7-azido-2-heptanol (R¹=4-methoxyphenyl-SO₂): from 4-methoxyphenyl methyl sulfone (1.53 g, 94% yield): ¹H-NMR (400 MHz, CDCl₃): δ7.85 (2H, d, J=8.8-Hz), 7.04 (2H, d, J=8.8-Hz), 4.13 (1H, m), 3.90 (3H, s), 3.24 (2H, t, J=6.8-Hz), 3.20 (1H, dd, J=8.8, 14.4-Hz), 3.14 (1H, dd, J=2.4, 14.4-Hz), 2.47 (3H, s), 1.57 (2H, m), 1.5˜1.3 (6H, m);

1-(2,4,6-trimethylphenylsulfonyl)-7-azido-2-heptanol ((R¹=2,4,6-trimethylphenyl-SO₂): from (2,4,6-trimethyl)phenyl methyl sulfone (1.30 g from 4.0 mMol reaction; 96%): ¹H-NMR (400 MHz, CDCl₃): δ6.99 (2H, s), 4.30 (1H, m), 3.49 (1H, d, J=2-Hz), 3.25 (2H, t, J=6.8-Hz), 3.18 (1H, d, J=1-Hz), 3.17 (1H, s), 2.66 (6H, s), 2.31 (3H, s), 1.59 (2H, m), 1.5˜1.3 (6H, m);

1-(morpholinosulfonyl)-7-azido-2-heptanol (R¹=O(CH₂CH₂)₂N-SO₂): from 1-morpholino methylsulfonamide (1.36 g from 10 mMol reaction, 89%): ¹H-NMR (400 MHz, d₆-DMSO): δ4.99 (1H, d, J=6.4 Hz), 3.88 (1H, m), 3.62 (4H, t, J=4.8-Hz), 3.32 (2H, t, J=6.8-Hz), 3.20˜3.15 (6H, overlap), 1.53 (2H, m), 1.46˜1.25 (6H, m);

1-(methylsulfonyl)-7-azido-2-heptanol (R¹=CH₃-SO₂): from dimethylsulfone; colorless oil (880 mg, 75%): ¹H-NMR (400 MHz, d₆-DMSO);

1-cyano-7-azido-2-heptanol (R¹=CN): from acetonitrile;

1-(methylsulfonyl)-7-azido-2-heptanol (R¹=CH₃-SO₂): from dimethylsulfone; colorless oil (880 mg, 75%): ¹H-NMR (400 MHz, CDCl₃): δ4.29 (1H, m), 3.28 (2H, t, J =7.2 Hz), 3.17 (1H, dd, J=9.6, 14.4 Hz), 3.07 (1H, dd, J=1.2, 14.4 Hz), 3.02 (3H, s), 2.90 (1H, d, J=3.6), 1.35˜1.7 (8H, m);

1-cyano-7-azido-2-heptanol (R¹=CN): from acetonitrile; colorless oil (320 mg, 0.98 mMol, 98%). ¹H-NMR (400 MHz, CDCl₃): δ5.18 (1H, d, J=5 Hz), 3.69 (1H, m), 3.32 (2H, t, J=6 Hz), 2.60 (1H, dd, J=4.8, 16.4 Hz), 2.51 (1H, dd, J=6.4, 16.4 Hz), 1.55 (2H, m), 1.42 (2H, m), 1.30 (4H, m);

1-(N,N-diethylaminosulfonyl)-7-azido-2-heptanol; from N,N-diethyl methane-sulfonamide, colorless oil [0.471 g (1.6 mMol) from 4.0-mMol reaction, 40% yield]; ¹H-NMR (400 MHz, CDCl₃): δ1.20 (6H, t, J=7.2 Hz), 1.41 (6H, m), 1.59 (2H, m), 2.95; and (2H, m), 3.26 (6H, m), 3.39 (1H, d, J=2.2 Hz), 4.15 (1H, m).

### Preparation B

### Preparation of Azido-Linker Chloroformates and Succinimidyl Carbonates

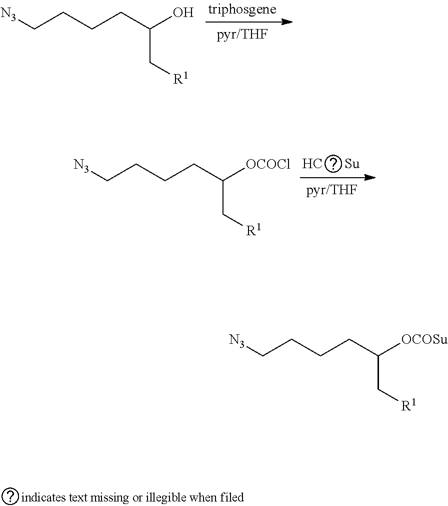

Pyridine (160 μL) was added dropwise to a stirred solution of the azidoalcohol (Preparation A, 1.0 mMol) and triphosgene (500 mg) in 15 mL of anhydrous THF. The resulting suspension was stirred for 10 minutes, then filtered and concentrated to provide the crude chloroformate as an oil.

Pyridine (300 μL) was added dropwise to a stirred solution of the above chloroformate and N-hydroxysuccinimide (350 mg) in 15 mL of anhydrous THF. The resulting suspension was stirred for 10 minutes, then filtered and concentrated to provide the crude succinimidyl carbonate. Purification by silica gel chromatography provided the purified product as an oil which typically spontaneously crystallized. Recrystallization could typically be effected using ethyl acetate/hexane.

### Preparation C

### Preparation of BOC-Amino-Linker Succinimidyl Carbonates

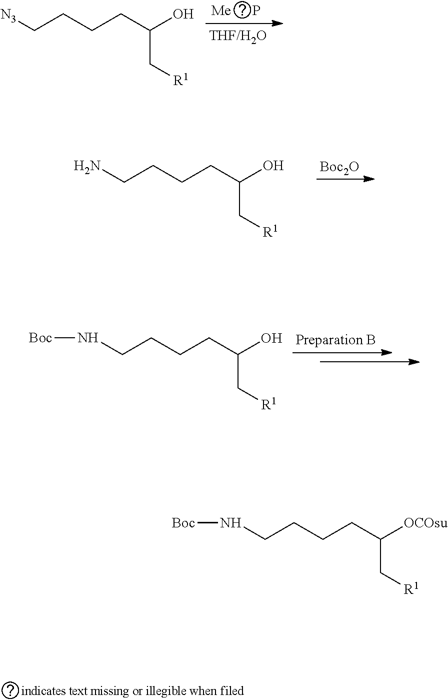

A stirred solution of an azido-linker alcohol of Preparation A (1 mMol) in 1 mL of tetrahydrofuran (THF) was treated with a 1.0 M solution of trimethyl-phosphine in THF (1.2 mL) for 1 hour at ambient temperature. Water (0.1 mL) was added, and the mixture was allowed to stir for an additional 1 hour, then the mixture was evaporated to dryness using a rotary evaporator. The residue was dissolved in ethyl acetate, washed with water and brine, then was dried over MgSO₄, filtered, and evaporated to provide the amino-alcohol.

A solution of the amino-alcohol (1.0 mMol) in 2 mL of THF was treated with di-tert-butyl dicarbonate (1.5 mMol) for 1 hour, and then evaporated to dryness. The residue was dissolved in ethyl acetate, washed with water and brine, then was dried over MgSO₄, filtered, and evaporated to provide the product. Chromatography on silica gel using a gradient of ethyl acetate in hexane provided the purified BOC-amino-alcohol.

BOC-amino alcohols were converted into the chloroformates and succinimidyl carbonates using the methods of Preparation B above.

### Preparation D

### Preparation of Bifunctional Crosslinkers of Formula (1) wherein X²=azide, R²=H, and X³=(CH₂CH₂O)ₙCH₂CH₂—N₃

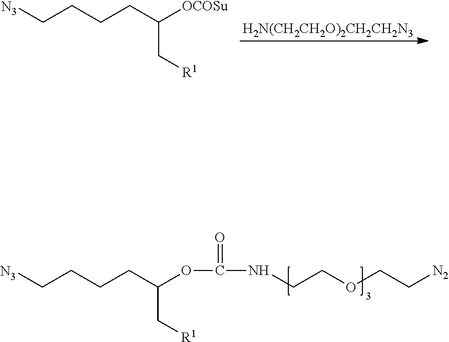

A solution of the succinimidyl carbonate of Preparation B (0.27 mMol) in 2 mL of acetonitrile was treated with 11-azido-3,6,9-trioxaundecan-l-amine (65 mg, 0.30 mMol) for 10 min at ambient temperature. After evaporation of the solvent, the residue was dissolved in 1 mL of CH₂C1₂ and chromatographed on a 4-g column of silica gel using a step gradient of hexane, 3:1 hexane/ethyl acetate, 1:1 hexane/ethyl acetate, and 1:2 hexane/ethyl acetate. The product-containing fractions were pooled and evaporated to provide the product.

### Preparation E

### Crosslinker Reagents of Formula (2) wherein Q=C(CH₂)₄, m is 0, t is 4, W is O(CO)NH, and X² is Azide

In a typical example, a solution of 25 μMol of the azido-linker-HSC (Preparation B) in 1 mL of acetonitrile was added to 5 μMol (100 mg) of 20-kDa 4-arm PEG-tetraamine hydrochloride in 1 mL of water and 40 μL of 1.0 M NaHCO₃. After 1 h at ambient temperature, TNBS assay indicated <2% of the amine groups remained, and the solution was dialyzed against 1 L of 50% methanol followed by 1 L of methanol (12 kDa cutoff membrane). After evaporation, the residue (109 mg) was dissolved in 2.12 mL of sterile-filtered 10 mM NaOAc, pH 5.0, and stored frozen at −20° C. The azide concentration was determined spectrophotometrically by reaction with DBCO acid.

### Preparation F

### Crosslinker Reagents of Formula (2) wherein Q=C(CH₂)₄, m is 0, t is 4, W is O(CO)NH, and X² is Amine

In a typical example, a solution of 26 mg (50 μMol) of the BOC-amino-linker-HSC (Preparation C), 200 mg of 20-kDa 4-arm PEG-tetraamine hydrochloride (10 μMol; 40 μMol amine), and 17 μL (100μMol) of diisopropylethylamine in 2 mL of tetrahydrofuran was stirred for 1 h at ambient temperature. TNBS assay indicated <1% of the amine groups remained, and the product was precipitated by slow addition of 10 mM of methyl t-butyl ether. The BOC-protected product was collected by vacuum filtration, washed with MTBE, dried, and stored frozen at −20° C.

The BOC-protected product (100 mg, 5 μMol) was dissolved in 2 mL of CF₃CO₂H, kept for 1 h, and then evaporated to dryness. The residue was washed twice with 5 mL portions of ether, then dissolved in 2 mL of THF and precipitated with 10 mL of MTBE to provide the PEG-(linker-amine)₄ as the trifluoroacetate salt. TNBS assay indicated 91% of the theoretical amine content by weight.

### Preparation G

### Crosslinker Reagents of Formula (2) wherein Q=C(CH₂)₄, m is 0, t is 4, W is O(CO)NH, and X² is Other Than Azide or Amine

PEG-linker-X² crosslinkers wherein X² is other than azide or NH₂ may be prepared by derivitization of the PEG-linker-amine crosslinkers of Preparation F using the appropriate reagents. Thus, crosslinkers wherein X²=SH may be prepared by reaction with a reagent such as the succinimidyl ester of 3-(2-pyridyldithio)propionate so as to prepare the intermediate 2-pyridyldisulfide, followed by reduction to the thiol using standard reagents like phosphines (TCEP, trimethylphosphine, triphenylphosphine, etc.). Reaction with a reagent such as the succinimidyl ester of 3-(maleimido)propionate will provide the crosslinkers wherein X²=maleimide.

### Preparation H

### Preparation of M-Tₓ wherein M=PEG, and X²=Cyclooctyne

PEG₂₀ kDa-(DBCO)₄: A 60 mM solution of freshly chromatographed DBCO-NHS (Click Chemistry Tools) in acetonitrile (0.5 mL, 30 μMol, 1.5 eq) was added to a solution of 20 kDa 4-arm PEG-amine hydrochloride (pentaerythritol core, JenKem Technologies; 100 mg, 5 μMol), and diisopropylethylamine (0.010 mL, 57 μMol) in acetonitrile (1 mL). After stirring 2 h at ambient temperature, the mixture was evaporated to dryness under reduced pressure. The residue was dissolved in 50% aqueous methanol (4 mL) and dialyzed against 50% aqueous methanol followed by methanol. After evaporation, the residue (100 mg) was dissolved in water to give a 50 mg/mL stock (10 mM DBCO by spectrophotometric assay), which was stored frozen at −20° C.

PEG₄₀ kDa-(DBCO)₈: One mL of 40 mM solution (40 μMol) of DBCO-NHS in THF was added to a solution of 168 mg (4.2 μMol) of 40-kDa 8-arm PEG-amine hydrochloride (tripentaerythritol core, JenKem Technologies) and 12.9 μL diisopropylethylamine (74 μMol) in 0.6 mL of ACN, and the mixture was kept at ambient temperature overnight. The reaction mixture was dialyzed against 2 L of 50% methanol followed by 1 L of methanol. After evaporation, the residue (149 mg) was dissolved in 1.49 mL water and stored frozen at −20° C. The DBCO concentration determined spectrophotometrically was 16 mM.

PEG₄₀ kDa(BCN)₈: A solution of 200 mg of 40 kDa 8-arm PEG-amine.HCl (JenKem Technologies; 40 μMol NH₂), 20 mg of BCN p-nitrophenyl carbonate (SynAffix; 63 μMol), and 20 μL of N,N-diisopropylethylamine (115 μMol) in 2 mL of DMF was stirred 16 h at ambient temperature. After quenching with 0.5 mL of 100 mM taurine in 0.1 M KPᵢ, pH 7.5, for 1 h, the mixture was dialyzed sequentially against water, 1:1 methanol/water, and methanol using a 12 kDa membrane. After evaporation, the residue was dissolved in 2 mL of THF and precipitated with 10 mL of methyl ^{t}butyl ether. The product was collected and dried (190 mg).

### Preparation I

### Preparation of Crosslinker of Formula (2)

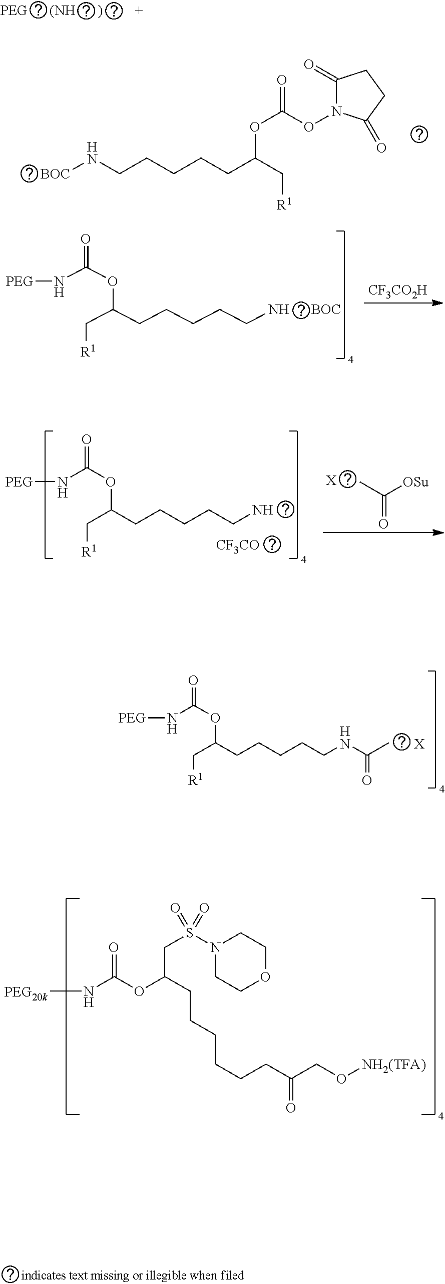

One method for preparation of multi-arm crosslinkers of formula (2) is illustrated wherein R¹=4-morpholino-SO₂, R²=H, n=0, one R⁵=h and the other R⁵ =(CH₂)₅NH—CO—CH₂O—NH₂, W═O(C═O)NH, Q=C(CH₂)₄, m˜110, s=2, and t=4. A solution of 4-arm 20 kDa PEG-tetraamine hydrochloride (200 mg, 10 uMol, JenKem Technologies), 7-(^{t}BOC-amino)-1-(4-morpholinosulfonyl)-2-heptyl succinimidyl carbonate (26 mg, 50 uMol), and N,N-diisopropylethylamine (17 uL, 100 uMol) in 2 mL of THF was kept for 1 h. The product was precipitated by addition of 10 mL of methyl ^{t} butyl ether (MTBE) and dried. Analysis by TNBS assay indicated 99% derivitization of PEG amine groups. A solution of PEG₂₀ kDa-[NHCO₂—CH(CH₂SO₂N(CH₂CH₂)₂O))(CH₂)₅NH^{t}BOC]₄ (94 mg) in 1:1 CF₃CO₂H/CH₂Cl₂ (2 mL) was kept at room temperature for 1.5 h then concentrated to dryness under vacuum. The resulting residue was dissolved in 2 mL of MeOH and precipitated with Et₂O (15 mL) to give the product (36 mg) as a white solid. A solution of PEG₂₀ kDa-[NHCO₂-CH(CH₂SO₂N(CH₂CH₂)₂O))(CH₂)₅NH₃⁺CF₃CO₂^{-]}₄ (34 mg, 0.0068 mMol (NH₂), 1 equiv) was treated with a solution of 2,5-dioxopyrrolidin-1-yl 2-(tert-butoxycarbonylaminooxy)acetate (2.35 mg, 0.0082 mMol, 1.2 equiv) and DIPEA (0.0012 mL, 0.9 mg, 0.0070 mMol, 1 equiv). The resulting mixture was kept at room temperature for 2 h then assessed for free amines by TNBS assay: A sample of the reaction mixture (0.020 mL) was incubated in 100 mM pH 9.4 borate buffer (1 mL) containing 0.04% w/v picrylsulfonic acid. The absorbance of this solution was monitored at 420 nm until stable (˜1 h) and compared to a reaction containing PEG₂₀ k-[SO₂Morph-linker-NH₂.TFA]₄ at the same concentration. Less than 2% amines remained. The reaction mixture was then diluted with Et₂O (15 mL) and the precipitated product was collected by filtration to give the product (29 mg) as a white solid. A solution of PEG₂₀ kDa-[NHCO₂-CH(CH₂SO₂N(CH₂CH₂)₂O))(CH₂)₅NH—CO—CH₂ONH^{t}BOC]₄ (39 mg) in 1:1 CF₃CO₂H/CH₂Cl₂ (2 mL) was kept at room temperature for 1.3 h then concentrated to dryness under vacuum. The resulting residue was triturated with Et₂O (2×10 mL) to give the product (29 mg) as a white solid.

### Preparation J

### Preparation of M Having Two Reactive Groups

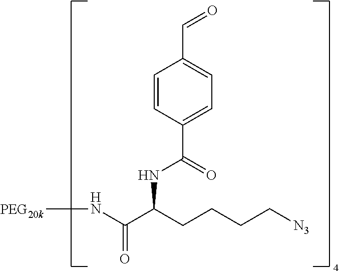

One method for the preparation of a multi-arm M wherein each arm is terminated with an adapter unit comprising two differentially-reactive groups is illustrated by preparation of a 4-arm 20-kDa PEG having azide and aldehyde groups.

(S)-6-azido-2-(4-formylbenzamido)hexanoic acid. A solution of Boc-Lys(N₃)—OH (Anaspec, 109 mg, 0.4 mMol) in 1:1 DCM:TFA (2 mL) was kept at room temperature for 1.5 h then concentrated to dryness under vacuum to give H-Lys(N₃)—OH (95 mg, 83%) as a white solid (TFA salt). A solution of H-Lys(N₃)—OH (19 mg, 0.066 mMol, 1 equiv) and DIPEA (0.035 mL, 26.0 mg, 0.20 mMol, 3 equiv) in DMF with 20% water (1.2 mL) was treated with a solution of 2,5-dioxopyrrolidin-1-yl 4-formylbenzoate (49.3 mg, 0.20 mMol, 3 equiv) in DMF (2 mL). The reaction was allowed to stir for 20 h prior to dilution with EtOAc (15 mL). The resulting mixture was extracted with 0.25 M NaHCO₃ (3×6 mL). The combine NaHCO₃ extracts were acidified to pH 2.5 with using 6 N HCl and extracted with EtOAc (4×6 mL). The combine EtOAc extracts were washed with water (3×5 mL), then brine (2 mL), and dried over MgSO₄ to give a white solid (30.4 mg). This material was further purified by C18 HPLC 20-85% ACN with 0.1% TFA linear gradient elution (5 mL/min) as the mobile phase. The combine product containing fractions were concentrated under vacuum to 50% of their original volume then extracted with EtOAc (5×10 mL). The EtOAc extracts were washed with water (5×10 mL) then brine (5 mL) and concentrated to dryness to give (S)-6-azido-2-(4-formylbenzamido)hexanoic acid (10.2 mg, 52%) as a clear oil. C18 HPLC 20-100% ACN with 0.1% TFA linear gradient elution RV=5.8 mL.

(S)-2,5-dioxopyrrolidin-1 -yl 6-azido-2-(4-formylbenzamido)hexanoate. A solution of N,N′-dicyclohexylcarbodiimide (6.81 mg, 0.033 mMol, 1 equiv) in THF (0.7 mL) was added to a solution of (S)-6-azido-2-(4-formylbenzamido)hexanoic acid (10.0 mg, 0.034 mMol, 1 equiv) and N-hydroxysuccinimide (3.8 mg, 0.033 mMol, 1 equiv) in THF (0.7 mL) at 4° C. The resulting mixture was kept at 4° C. for 17 h then filtered. The filtrate was concentrated to dryness to give 18 mg of residue. This crude material (S)-2,5-dioxopyrrolidin-1-yl 6-azido-2-(4-formylbenzamido)hexanoate (13 mg, max yield) was used to acylate PEG₂₀ k-(NH₂)₄ without further purification. C18 HPLC 20-100% ACN with 0.1% TFA linear gradient elution RV=6.8 mL.

PEG₂₀ k-[Lys(N₃)-CHO]₄. A solution of PEG₂₀ k(NH₂.HCl)₄ (JenKem, 27 mg, 0.0054 mMol (NH₂), 1 equiv) and DIPEA (0.0028 mL, 3 mg, 0.0162 mMol, 3 equiv) in acetonitrile (0.5 mL) was treated with a solution of (S)-2,5-dioxopyrrolidin-1-yl 6-azido-2-(4-formylbenzamido)hexanoate (˜6.5 mg, 0.0162 mMol, 3 equiv) in DMF (0.5 mL). The resulting mixture was kept for 2 h then assessed for free amines by TNBS assay: A sample of the reaction mixture (0.018 mL) was incubated in 100 mM pH 9.4 borate buffer (1 mL) containing 0.04% w/v picrylsulfonic acid. The absorbance of this solution was monitored at 420 nm until stable (˜1 h) and compared to a reaction containing PEG₂₀ k-(NH₂.HCl)₄ at the same concentration. Less than 2% amines remained. The reaction mixture was filtered to remove a small amount of insoluble material then it was then diluted with Et₂O (15 mL) and the precipitated product was collected by filtration to give PEG₂₀ k-[Lys(N₃)-CHO]₄ (26 mg) as a white solid.

### Preparation K

### Preparation of X-Linker-Drugs

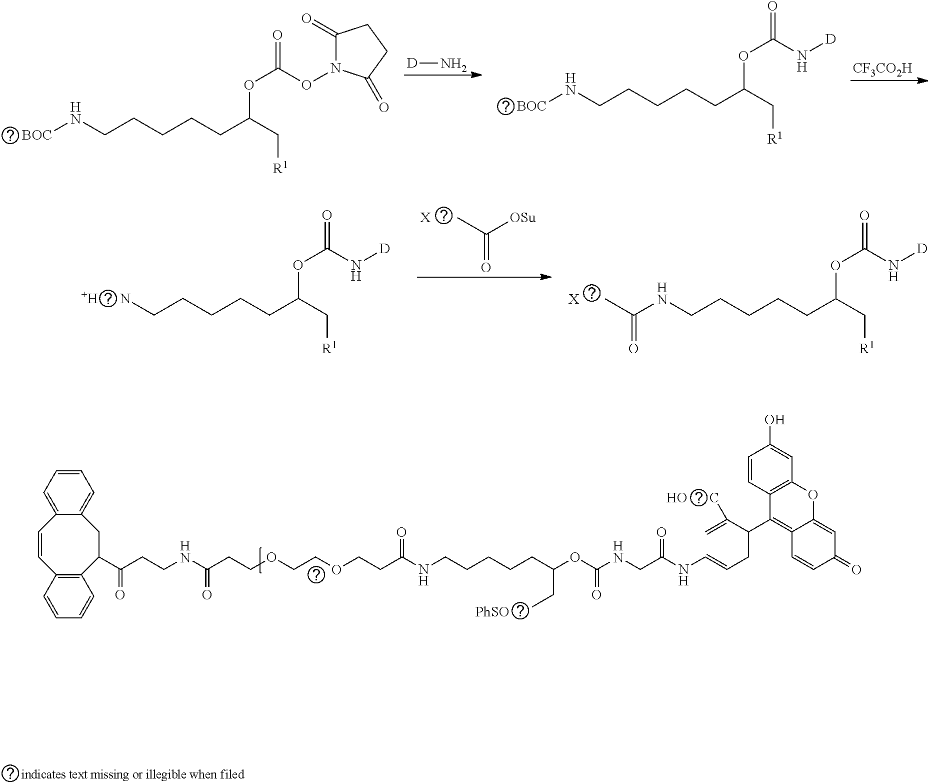

One method for formation of X-linker-Drugs useful in attaching to the sealants of the invention is illustrated using D-NH₂=5-(aminoacetamido)fluorescein as a model drug. A solution of 5-(aminoacetamido)fluorescein (Invitrogen, 0.1 mL, 21.7 mM, 0.0022 mMol, 1 equiv) in DMF was mixed with a solution of the linker of formula (1b) wherein R¹=phenylsulfonyl, R²=H, n=0, one R⁵=H, the other R⁵=(CH₂)₅NH^{t}BOC, and X⁴=O-succinimidyl (0.087 mL, 25 mM, 0.0022 mMol, 1 equiv). The resulting mixture was kept at room temperature for 1.5 h then it was acidified with 0.001 N HCl (5 mL), a yellow precipitate forms, and extracted with EtOAc (2×5 mL). The combine EtOAc extracts were washed with water (2×3 mL), then brine (2 mL), then dried over MgSO₄, and concentrated to dryness under vacuum to give ˜2 mg of a yellow residue. This material was treated with 1:1 DCM:TFA (1 mL) for 1 h at room temperature. The resulting mixture was then concentrated to dryness under vacuum to give the amine intermediate as a yellow residue. This material was dissolved in acetonitrile (1 mL) to give a solution containing 1.4 mM (0.0014 mMol, 64% yield) fluorescein based on ε₄₉₅=80000 M⁻¹ cm⁻¹. C18 HPLC 20-100% ACN with 0.1% TFA linear gradient elution RV=5.3 mL. Replacement of the 5-(aminoacetamido)fluorescein with an amine-containing drug allows for analogous preparation of linker-drug units. A solution of the linker-drug unit in acetonitrile (1 mL, 1.4 mM, 0.0014 mMol, 1 equiv) was treated with a solution of DBCO-PEG₄-NHS ester (Click Chemistry Tools) in acetonitrile (0.0372 mL, 37.6 mM, 0.0014 mMol, 1 equiv) and a solution of N,N-diisopropylethylamine (DIPEA) in acetonitrile (5.74 mM, 0.50 mL, 0.0028 mMol, 2 equiv). The resulting mixture was kept at room temperature for 2.5 h then acidified with 0.001 N HCl (20 mL) and extracted with EtOAc (3×5 mL). The combine EtOAc extracts were washed with water (3×5 mL), then brine (2 mL), and concentrated to dryness. The resulting residue was dissolved in 0.3 mL DMF to give a solution containing 2.8 mM fluorescein (0.00084 mMol, 60%) based on ε₄₉₅=80000 M⁻¹ cm⁻¹. C18 HPLC 20-100% ACN with 0.1% TFA linear gradient elution RV =7.5 mL. This method can be used to prepare analogous X-linker-drugs through appropriate choice of the X-NHS reagent.

### Preparation L

### Preparation of a Fluorescent Gel Degradation Probe (DEAC-DBCO)

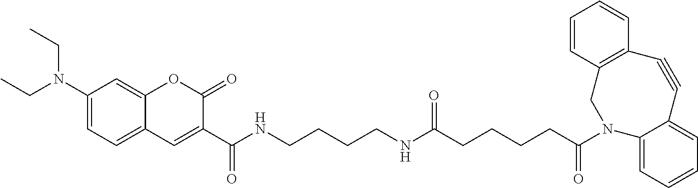 [SMILES: CCN(CC)c1ccc2cc(C(=O)NCCCCNC(=O)CCCCC(=O)N3Cc4ccccc4C#Cc4ccccc43)c(=O)oc2c1]

To a solution of 7-(diethylamino)coumarin-3-carboxylic acid N-succinimidyl ester (Fluka 36801, 20.9 mg, 0.056 mMol, 1 equiv) in DMSO (1.4 mL) was added a solution of N-Boc-1,4-diaminobutane (Sigma 15404, 11.7 mg, 0.062 mMol, 1.1 equiv) in DMSO (0.72 mL), followed by Et₃N (24 μL, 17.4 mg, 0.17 mMol, 3 equiv). The resulting mixture was allowed to stir at room temperature for 1.5 h, at which time the reaction was complete by TLC (Silica, 2:1 EtOAc:Hexanes, R_{f}(coumarin-NHS)=0.29, R_{f}(BOC-product)=0.43). The reaction mixture was then diluted with EtOAc (20 mL), washed with water (5×4 mL), then brine (2 mL), then dried over solid MgSO₄, filtered through silica, and concentrated to dryness under reduced pressure. The resulting residue was dissolved in DCM (1 mL), treated with TFA (1 mL) for 1 h at room temperature, at which time the reaction was complete by TLC (Silica, EtOAc, R_{f}(BOC)=0.63, R_{f}(amine)=0). The reaction mixture was concentrated to dryness under reduced pressure. The resulting residue was triturated with Et₂O (3×5 mL portions) then dried under vacuum to give DEAC-NH₂ (26.3 mg, 0.059 mMol, 105%) as a yellow solid (TFA salt). C₄ HPLC 0-100% ACN 0.1% TFA linear gradient elution (RVₐₘᵢₙₑ=5.8 mL). A solution of DEAC-NH₂(TFA) (3.88 mg, 0.0087 mMol, 1 equiv) in DMF (0.5 mL) was treated with DIPEA (0.0033 mL, 2.5 mg, 0.0019 mMol, 2 equiv) and a solution of DBCO-NHS ester (Click Chem. Tools A102, 43.6 mM, 0.200 mL, 0.0087 mMol, 1 equiv). The resulting mixture was allowed to stir overnight (a small amount of DBCO-NHS remains as determined by C18 HPLC) then treated with 10 mM taurine in pH 7.5 HEPES. The resulting mixture was allowed to stir for 4 h then it was diluted with 0.5 N NaHCO₃ (10 mL) and extracted with EtOAc (3 x 6 mL). The combine EtOAc extracts DEAC-DBCO (4.7 mg, 84%). This material was dissolved in DMF (0.7 mL) to give a 7.9 mM solution based on ε₄₃₀=44800 M⁻¹cm⁻¹.

### Preparation M

### Macromonomer with N₃ and Succinimidyl Ester End Groups

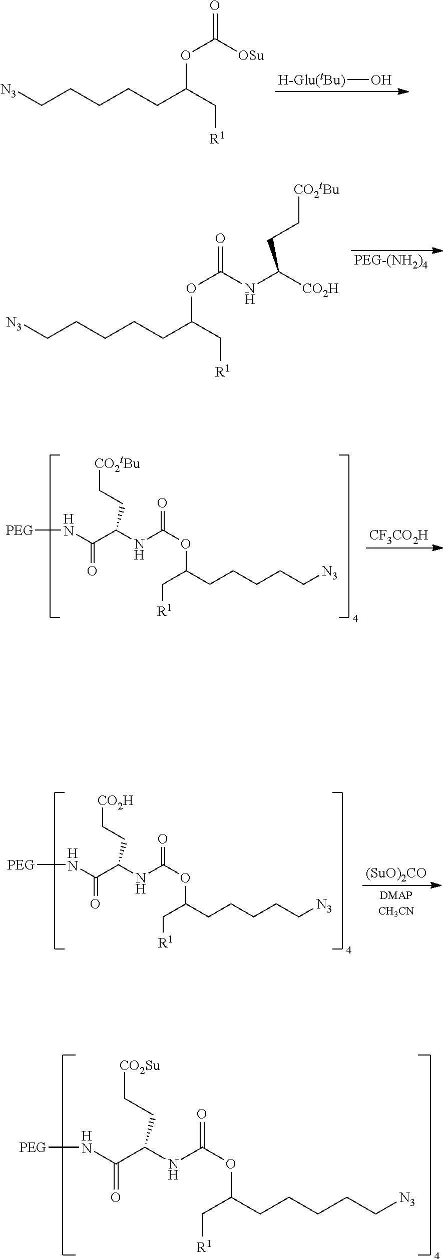

N-{7-Azido-1-[N-methyl-N-(2-methoxyethyl)aminosulfonyl]-2-heptyloxycarbonyl}-Glu(OtBu)-OH. N,N-Diisopropylethylamine (164 μL, 942 μMol) and a solution of 7-azido-1-[N-methyl-N-(2-methoxyethyl)aminosulfonyl]-2-heptyl succinimidyl carbonate (385 mg, 857 μMol) in 4 mL of acetonitrile were added to a suspension of H-Glu(OtBu)-OH (191 mg, 942 μMol) in 4 mL of acetonitrile. Suspended H-Glu(OtBu)-OH did not dissolve upon addition of reagents; thus, 8 mL of DMF was added. The solid did not immediately dissolve. Next, 2 mL of water was added, and the suspended solid dissolved within 2 min. After 30 min at ambient temperature the reaction was judged to be complete by TLC analysis, and the mixture was partitioned between 150 mL of 1:1 EtOAc:KHSO₄ (½ sat aq). The layers were separated, and the organic phase was successively washed with KHSO₄ (2.5% aq), water, and brine (1×100 mL each). The organic layer was then dried over MgSO₄, filtered, and concentrated by rotary evaporation. The resulting crude colorless oil was purified by silica gel column chromatography (4 g) eluting with dichloromethane (40 mL) followed by a gradient of acetone in dichloromethane: 15% (40 mL), 30% (40 mL), and 65% (40 mL). Mixed fractions were rechromatographed eluting with dichloromethane (30 mL) followed by a gradient of acetone in dichloromethane: 3% (30 mL), 6% (30 mL), 9% (30 mL), 12% (30 mL), and 15% (30 mL). Clean product containing fractions from both columns were combined and concentrated to provide 316 mg (69%) of the title compound as a thick colorless oil.

N-{7-Azido-1-[N-methyl-N-(2-methoxyethyl)aminosulfonyl]-2-heptyloxycarbonyl}-Glu(OtBu)-OSu. N,N′-Disuccinimidyl carbonate (196 mg, 764 μMol) and 4-(dimethylamino)pyridine (0.20 M in acetonitrile, 0.30 mL, 60 μMol) were successively added to a solution of N-{17-azido-1-[N-methyl-N-(2-methoxyethyl)-aminosulfonyl]-2-heptyloxycarbonyl}-Glu(OtBu)-OH (316 mg, 589 μMol) in 5.6 mL of acetonitrile. The reaction mixture was stirred at ambient temperature while monitoring progress by TLC. After 15 min, the reaction was judged to be complete, and the mixture was partitioned between 200 mL of 1:1 EtOAc:NaHCO₃ (½ sat aq). The layers were separated, and the organic phase was successively washed with water, KHSO₄ (2.5% aq), water, and brine (1×100 mL each). The organic layer was then dried over MgSO₄, filtered, and concentrated by rotary evaporation and high vacuum to provide 353 mg (94% crude) of the crude title compound as a colorless oil. The product was used without further purification.

{N-[7-Azido-1 -(N-methyl-N-(2 -methoxyethyl)aminosulfonyl)-2-heptyloxycarbonyl]-Glu(OtBu)}₄-PEG₂₀ kDa. N,N-Diisopropylethylamine (164 μL, 942 μMol) and N-{17-azido-1-[N-methyl-N-(2-methoxyethyl)aminosulfonyl]-2-heptyloxycarbonyl}-Glu(OtBu)-OSu (137 μM in MeCN, 2.85 mL, 390 μMol) were successively added to a solution of PEG₂₀ kDa-(NH₂.HCl)₄ in 12 mL of acetonitrile. The reaction mixture was stirred at ambient temperature while monitoring progress by TLC. After 20 min, the starting succinimidyl ester was not observed by TLC. More N-{7-azido-1-[N-methyl-N-(2-methoxyethyl)aminosulfonyl]-2-heptyloxycarbonyl}-Glu(OtBu)-OSu (137 μM in MeCN, 0.57 mL, 78 nMol) was added, and the reaction mixture was stirred for 1.7 h more (2 h total). Acetic anhydride (28 μL, 0.30 mMol) was added to cap any unreacted amines, and stirring was continued for 20 min more. The reaction mixture was concentrated to 6 mL then added to 80 mL of tent-butyl methyl ether. The resulting suspension was stirred for 30 min then vacuum filtered. Solids were washed with tert-butyl methyl ether (3 x 20 mL) then dried under vacuum to provide 1.48 g (89%) of the title compound as a white powder.

{N-[7-Azido-1-(N-methyl-N-(2-methoxyethyl)aminosulfonyl)-2 -heptyloxycarbonyl]-Glu}₄-PEG₂₀ kDa. Trifluoroacetic acid (7 mL) was added to a solution of {N-[7-azido-1-(N-methyl-N-(2-methoxyethyl)aminosulfonyl)-2-heptyloxycarbonyl]-Glu(OtBu)}₄-PEG₂₀ kDa (1.48 g, 67.0 μMol PEG) in 7 mL of dichloromethane. The reaction mixture was stirred at ambient temperature while monitoring progress by C18 HPLC. After 2 h, the reaction mixture was concentrated to dryness. The crude residue was redissolved in 8 mL of tetrahydrofuran then added dropwise to 80 mL of diethyl ether. The resulting suspension was stirred for 30 min then vacuum filtered. Solids were successively washed with diethyl ether (3×30 mL) and 30 mL of tert-butyl methyl ether then dried under vacuum to provide 1.34 g (91%) of the title compound as a white powder.

{N-[7-Azido-1-(N-methyl-N-(2-methoxyethyl)aminosulfonyl)-2-heptyloxycarbonyl]-Glu(OSu)}₄-PEG₂₀ kDa. N,N′-Disuccinimidyl carbonate (9.3 mg, 36 μMol) and 4-(dimethylamino)pyridine (0.2 M in MeCN, 14 μL, 2.8 μMol) were successively added to a solution of {IN-[7-azido-1-(N-methyl-N-(2-methoxyethyl)aminosulfonyl)-2-heptyloxycarbonyl]-Glu}₄-PEG₂₀ kDa (140 mg, 6.4 μMol PEG, 26 μMol CO₂H) in 1.4 mL of acetonitrile. The reaction mixture was stirred at ambient temperature while monitoring progress by C18 HPLC. After 45 min, the reaction mixture was added dropwise to 18 mL of tert-butyl methyl ether. The resulting suspension was stirred for 30 min, and the supernatant was decanted. The solid was resuspended in 10 mL of tert-butyl methyl ether then vacuum filtered. The solids were washed successively with 2-propanol (2×8 mL) and tert-butyl methyl ether (2×8 mL) then dried under vacuum to provide 122 mg (86%) of the title compound as a white powder.

### Preparation N

### PEG-octa(succinimidyl succinamidate) PEG-(NHCO—CH₂CH₂—COOSu)₈

A mixture of 8-arm 20-kDa PEG-octaamine.HCl (4.10 g, 205 μMol, 1640 μMol amine; tripentaerythritol core, JenKem), succinic anhydride (400 mg, 4000 μMol), and N,N-diisopropylethylamine (700 uL, 4000 μMol) in 40 mL of anhydrous acetonitrile was stirred for 30 min, at which time there were no remaining amine groups by TNBS assay. The polymer was precipitated by slow addition to 200 mL of stirred 2-propanol. The solid was collected by vacuum filtration and dried under vacuum (4.13 g). This material was dissolved in 20 mL of anhydrous acetonitrile and treated with disuccinimidyl carbonate (1.4 g, 5.5 mMol) and 4-(dimethylamino)pyridine (50 mg, 0.41 mMol) for 16 h. The solvent was evaporated under vacuum, and the residue was dissolved in 20 mL of THF and precipitated by slow addition to 150 mL of stirred MTBE. The solid was collected by vacuum filtration, washed once with 2-propanol, and dried under vacuum. The dried material was similarly precipitated once from 2-propanol, dried, then a second time from MTBE and dried to provide the product octa(succinimidyl ester) as a white solid, 4.0 g (90%). To analyze the product, a sample (2.7 mg) was reacted with 200 uL of 10 mM 4-nitrobenzylamine hydrochloride and 20 mM N,N-diisopropyl-ethylamine in 800 uL of acetonitrile for 30 min, and the mixture was analyzed by reversed-phase HPLC with integration of peaks detected at 275 nm, which indicated 7.9±0.2 HSE groups/PEG. The product was further analyzed using a published method (Gao, et al., Chemistry Central J. (2012) 6:142) that indicated 106 ±14% of the expected HSE content. Use of these materials rather than the ester-linked PEG-succinimidyl succinate (PEG-OCO—CH₂CH₂—COOSu)₈ or PEG-succinimidyl glutarate (PEG-OCO—CH₂CH₂CH₂—COOSu)₈ provide sealants that are resistant to hydrolytic degradation due to the lack of ester linkages in the final sealants.

### Preparation O

### PEG-(Linker-Succinimidyl Carbonate)₈

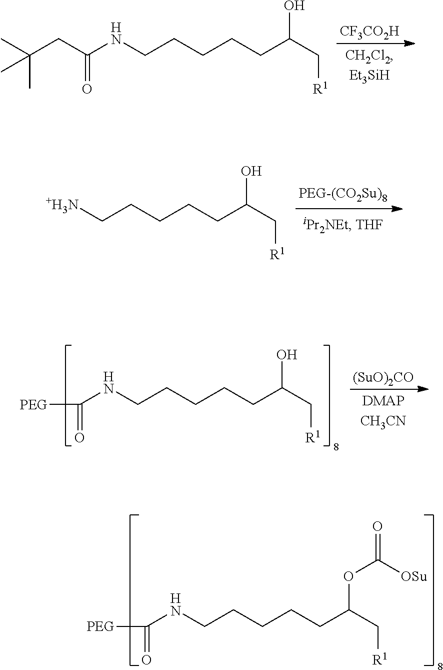

A general method for preparation of PEG-(linker-succinimidyl carbonate)₈ macromonomers is illustrated by the specific preparation of the compound wherein R¹ is SO₂N(Me)(CH₂CH₂OMe). 7-(BOC-amino)-1-(N-methyl-N-(2-methoxyethyl)-aminosulfonyl)-2-heptanol (192 mg, 500 μMol; Preparation C) was dissolved in 2 mL of 1:1 CH₂Cl₂/CF₃CO₂H+1% triethylsilane. After 30 min, the mixture was evaporated to dryness and the residue was washed 3x with ethyl ether. The residue was dissolved in 5 mL of THF and treated with 8-arm 20-kDa PEG-octa(succinimidyl succinate) (1.00 g, 50 μMol PEG, 400 μMol amine, Preparation N) and N,N-diisopropylethylamine (100 uL, 575 uMol). Reaction progress was monitored by assay of aliquots in the 4-nitrobenzylamine assay described above. After complete consumption of HSE groups (1 h), the mixture was added slowly to 100 mL of stirred 2-propanol and the precipitated product was collected, washed with MTBE, and vacuum dried to give 1.01 g (84% yield) of PEG-(linker-alcohol)₈.

The PEG-(linker-alcohol)₈ (1.0 g, 42 μMol PEG) was dissolved in anhydrous acetonitrile (5 mL) and treated with N,N′-disuccinimidyl carbonate (256 mg, 1000 μMol) followed by a solution of 4-(dimethylamino)pyridine (100 mg, 820 μMol) in 1 mL of acetonitrile. The resulting clear solution was stirred for 6 h, and then ether was added to precipitate the product. The precipitate was collected and dried, and the product was purified by repeated precipitations.

### Example 1

### Preparation of Degradable Sealants

Two solutions are prepared. In one method, the first solution comprises the polymer MX¹ₓ dissolved in water or buffer, optionally with a pharmaceutically acceptable excipient. The second solution comprises X²-(1′)-P, X²-1b′-D if present, where 1′ and 1b′ represent reacted forms of formulas 1 and 1b, and the crosslinker T in the appropriate molar ratios dissolved in water or buffer, optionally with a pharmaceutically acceptable excipient. The molar ratios are calculated to provide the desired loading of P and D groups and degree of gel crosslinking. If necessary, excess X¹ groups may be capped by inclusion of an appropriate capping reagent such that the total concentrations of X¹ and X² groups are equal in the final polymerization mixture. Alternately, excess X¹ and X² groups may be used in the final polymerization mixture if subsequent sealant layers are to be applied.

In a second method, the first solution comprises the macromonomer, M, comprising functional groups X¹ together with X²-(1′)-P and X²-(1b′)-D if present, dissolved in water or buffer, optionally with a pharmaceutically acceptable excipient. The second solution comprises the crosslinker T dissolved in water or buffer, optionally with a pharmaceutically acceptable excipient.

To prepare the sealant in either method, the appropriate amounts of the first and second solutions are mixed and applied to the site of the wound, suture, or anastomosis. The polymerization mixture is applied to the wound site and allowed to set. Application may be as a bulk liquid, for example by extrusion from a syringe onto the wound, or by aerosol, for example using a spray device. The two solutions may be premixed or mixed during application using a multi-channel device, for example a multi-barrel syringe wherein each barrel contains a component of the polymerization mixture and the reactive components are mixed upon extrusion into the syringe tip or needle.

### Example 2

### Preparation of Degradable Sealants wherein M and T=PEG, X¹=Cyclooctyne, X²=Azide, P=N-hydroxysuccinimidyl Carbonate, and D is Absent

A solution comprising an 8-arm PEG-(cyclooctyne)₈ (Preparation G) dissolved in water is mixed with a freshly prepared solution comprising an azide-linker-succinimidyl carbonate (Preparation D) and a 4-arm PEG-(linker-azide)₄ crosslinker (Preparation H) in 10 mM acetate buffer, pH 5 so as to form a polymerization mixture. The proportions of the three components are adjusted so as to provide the required tissue adhesion (controlled by the concentration of azide-linker-succinimidyl carbonate) and the mechanical properties of the sealant (controlled by the concentration of crosslinker and the total concentration of PEG). The polymerization mixture is applied to the site requiring sealing and allowed to set. Application may be as a bulk liquid, for example by extrusion from a syringe onto the wound or through application using a brush, or by aerosol, for example using a spray device. The two solutions may be premixed or mixed during application using a multi-channel device, for example a multi-barrel syringe wherein each barrel contains a component of the polymerization mixture and the reactive components are mixed upon extrusion into the syringe tip or needle.

### Example 3

### Preparation of Degradable Sealants wherein M and T=PEG, X¹ =N-hydroxysuccinimidyl Ester, X²=Thiol, P=N-hydroxysuccinimidyl Carbonate, and D is Absent

A solution comprising a 4-arm PEG-(N-hydroxysuccinimidyl ester)₄ is mixed with a 4-arm PEG-(linker-SH)₄ (Preparation J) and the resulting polymerization mixture is applied to the site requiring sealing to provide a degradable sealant wherein the sealant matrix is formed through thioester bonds. The proportion of the two components is adjusted such that the N-hydroxysuccinimidyl ester groups are present in molar excess over thiol groups, thus providing tissue adherence.

### Example 4

### Preparation of Degradable Sealants wherein M is albumin, T=PEG, X¹=Amine, X²=N-hydroxysuccinimidyl Ester, P=N-hydroxysuccinimidyl Ester, and D is Absent

A solution comprising an albumin is mixed with a freshly-prepared solution of PEG-(N-hydroxysuccinimidyl ester)ₓ wherein x=2-8 in buffer at a pH value between 6 and 8, and the resulting polymerization mixture is applied to the site requiring sealing.

### Example 5

### Degradable Sealant of Formula (2) wherein M=PEG, T=Trilysine, X¹=N-hydroxysuccinimidyl Ester, X²=Amine, and L₃-D is Absent

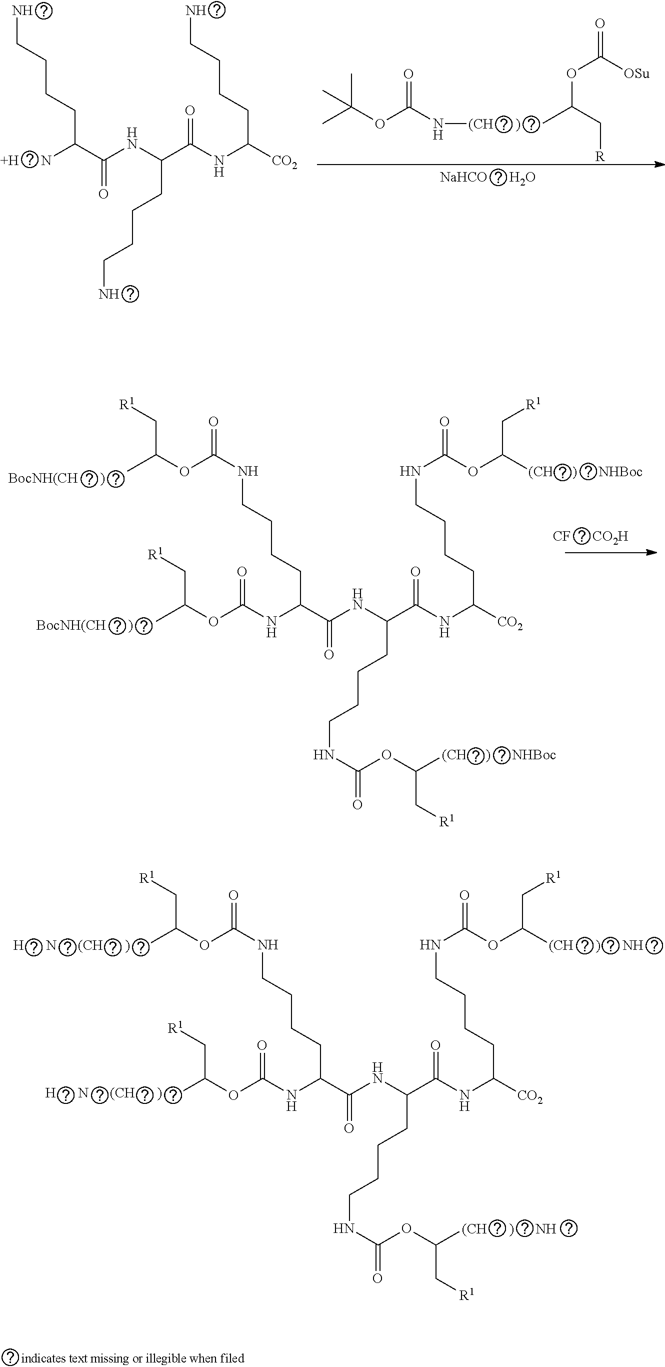

Trilysine is reacted with excess BOC-amino-linker-succinimidyl carbonate (Preparation E) to produce the (BOC-amino-linker-carbamoyl)₄-trilysine intermediate, which is dissolved in trifluoroacetic acid to remove the BOC protection and provide (amino-linker-carbamoyl)₄-trilysine as the trifluoroacetate salt. A solution of the (amino-linker-carbamoyl)₄-trilysine salt in aqueous buffer is mixed with a freshly-prepared aqueous solution of a 4-arm PEG-(succinimidyl ester)₄ such that the final pH is between 7 and 8, and the resulting polymerization mixture is applied to the site requiring sealing.

### Example 6

### Degradable Sealant of Formula (2) wherein M=poly(ethylene imine), T=PEG, X¹=Amine, X²=N-hydroxysuccinimidyl Carbonate, and L₃-D is Absent

In one method, a solution of poly(ethylene imine) is mixed with an azido-linker-succinimidyl carbonate to produce a polymer comprising azido-linker-carbamates. This polymer solution is mixed with a solution of a bifunctional PEG-cyclooctyne and applied to the site requiring sealing to produce a degradable sealant.

In a second method, a solution of poly(ethylene imine) is mixed with a solution comprising azido-linker-succinimidyl carbonate and a bifunctional PEG-cyclooctyne and applied to the site requiring sealing to produce a degradable sealant.

### Example 7

### Hydrogel Formation from M Having Two Differentially-Reactive Functional Groups

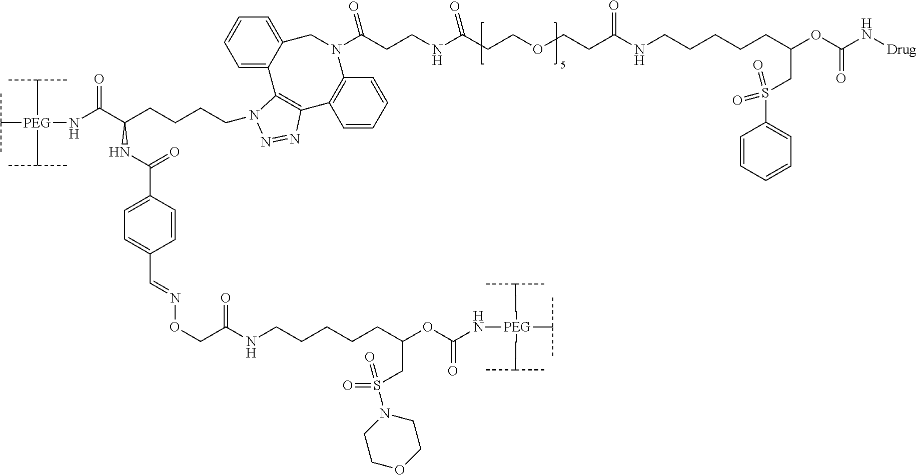

To illustrate one embodiment of the invention, a hydrogel containing 5% w/v total PEG was made by mixing an aqueous solution of PEG₂₀ k-[CHO-Lys(N₃)]₄ (100 mg/mL, 20 mM CHO and N₃, 0.025 mL, 0.0005 mMol, 1 equiv) with 0.5 M pH 4.5 acetate buffer (0.0106 mL, 0.5 M), a solution of AAF-(SO₂Ph-linker)-PEG4-DBCO in DMF (2.8 mM, 0.018 mL, 0.000050 mMol, 0.1 equiv), and a solution of DEAC-DBCO in DMF (7.9 mM, 0.0114 mL, 0.000090 mMol, 0.18 equiv). The resulting mixture was kept at room temperature for 20 min then an aqueous solution of PEG₂₀ k-[SO₂Morph-linker-O—NH₂.TFA]₄ (100 mg/mL, 20 mM 0-NH₂, 0.025 mL, 0.0005 mMol, 1 equiv) and an aqueous solution of aniline (pH 4.5, 1 M, 0.010 mL, 0.010 mMol, 20 equiv) were added. To form the sealant into a defined size and shape for further study, the resulting mixture was immediately placed into a 64 uL (9×1 mM) circular rubber perfusion chamber (Grace Bio-Labs) mounted on a silanized glass microscope slide, and allowed to cure overnight. In order to add tissue-attachment groups P to the above gel together with releasable drug D, a mixture of D-linker-PEG4-DBCO and P-DBCO or P-linker-DBCO would be used.

### Example 8

### Preparation of Multi-Layer PEG Hydrogels

A two-layer hydrogel was prepared as follows. A first 5% w/v hydrogel (A) comprising excess cyclooctyne groups was prepared by mixing PEG₄₀ kDa-(BCN)₈ (50 uL of 100 mg/mL in H₂O; 1000 nMol BCN), 0.1 M MES, pH 6.0 (60 uL), and 4-azidobutyryl-Lys(DNP)-OH (15 uL of 10 mM in MeOH; 150 nMol N₃), then adding PEG₂₀kDa-(NH—CO—O—CH(CH₂SO₂N(CH₂CH₂)₂O)(CH₂)₅N₃)₄ (25 uL of 100 mg/mL in H₂O; 500 nMol N₃), vortexing, centrifuging to remove air bubbles, and pipetting into gel molds. A second 5% w/v hydrogel (B) comprising excess azide groups was prepared by mixing PEG₄₀ kDa-(BCN)₈ (37.5 uL of 50 mg/mL in H₂O; 750 nMol BCN), 0.1 M MES, pH 6.0 (60 uL), and 4-azidobutyryl-AAF (2.5 uL of 30 mM in MeOH; 75 nMol N₃), then adding PEG₂₀ kDa-(NH—CO—O—CH(CH₂CN)(CH₂)₅N₃)₄ (50 uL of 100 mg/mL in H₂O; 1000 nMol N₃). Once the gels had set (30 minutes), they were removed from the molds and suspended in water. Junctions between gels were prepared by stacking one gel on the other followed by application of slight pressure to remove interfacial air and liquid. The junctions were allowed to sit for 1 hour, and then examined for adhesion by physical separation. Junctions prepared from equivalent disks, i.e., A-A and B-B, were found to readily separate into the intact component disks. The A-B junction prepared from complementary disks, however, was found the form a cohesive inseparable bond.

### Example 9

### In vitro Drug Release and Sealant Degradation

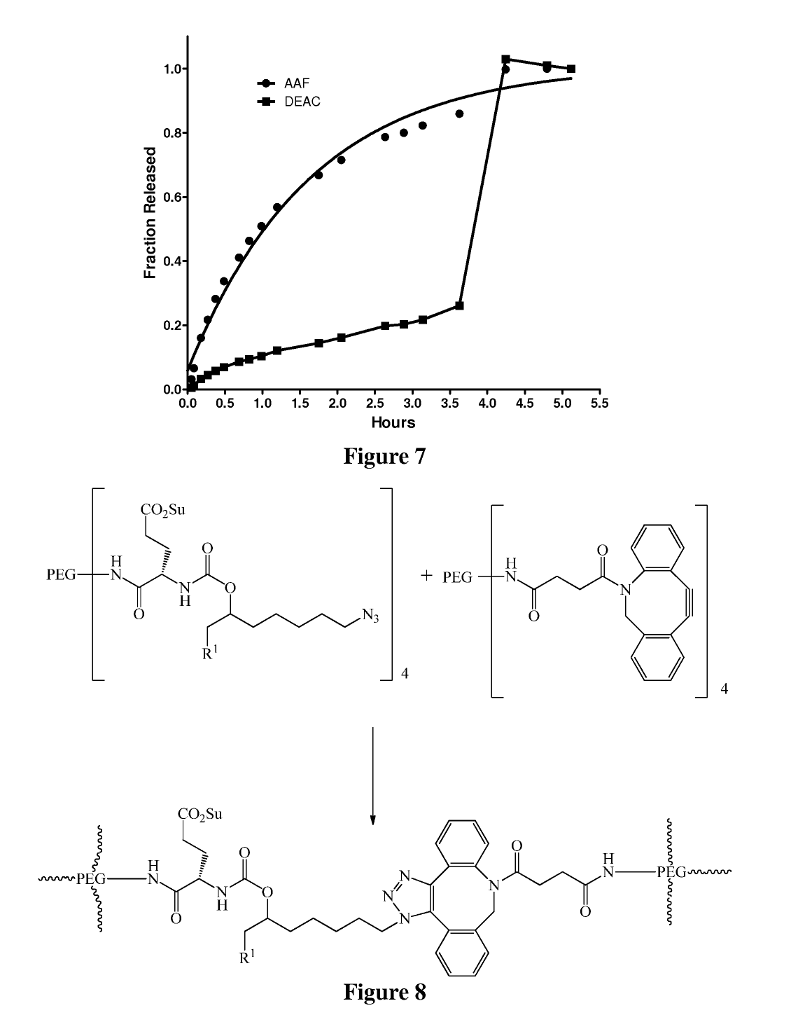

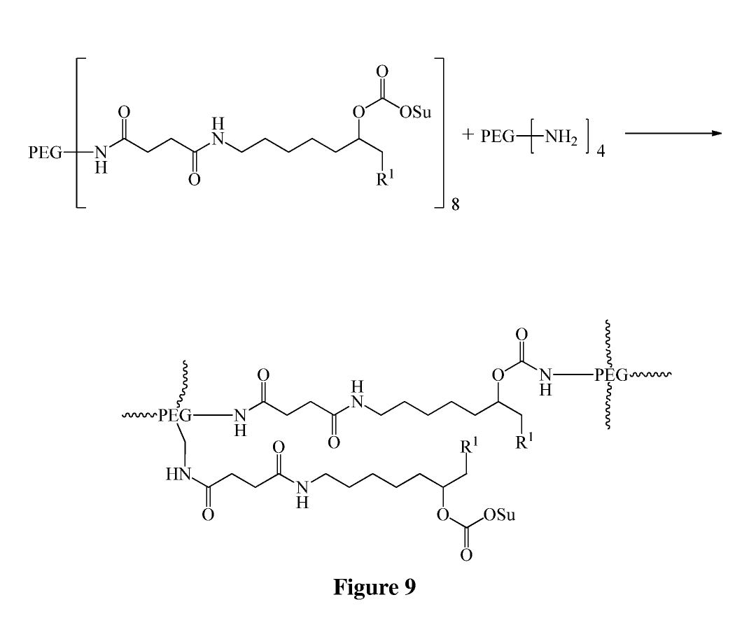

After curing for 18 h at room temperature the hydrogel disc from Example 7 was placed into 10 mL of 10 mM pH 5.5 Acetate (2×30 min) to remove aniline catalyst then it was placed into 2.5 mL of pH 9.4 borate buffer at 37° C. in a divided cuvette. The OD of the buffer was monitored over time to measure drug release (fluorescein (AAF) 495 nm, solid circles) or gel degradation (coumarin (DEAC) 430 nm, solid squares). At pH 9.4, drug release was observed with t_{1.2}=1 h, while gel degradation was minimal up to the degelation point at ˜4 h. As both rates have been shown to be first-order in hydroxide concentration, the rates observed at pH 9.4 can be translated into a half-life of 100 h for drug release and a degelation time of 400 h (16 days) at pH 7.4 as shown in FIG. 7.

### Example 10

### Amide-Linked Sealants

In one formulation, macromonomer solutions were prepared by dissolving PEG-(NHCO—CH₂CH₂—COOSu)₈ in 0.01 M phosphate, pH 5, and PEG-(NHCO₂—CH(CH₂R¹)—(CH₂)₅NH₂)₄.(CF₃CO₂H)₄ (Preparation I) in 0.1 M phosphate, pH 8. Sealants were prepared by mixing the two macromonomer solutions at appropriate volume ratios to provide the desired total PEG concentration, crosslinking density, and concentration of residual succinimidyl esters for tissue adhesion. For example, using macromonomers prepared using 20-kDa PEGs, mixing of equal volumes of the two 50 mg/mL macromonomer solutions provides a sealant having total PEG concentration of 5%, an average crosslinking density of 4 crosslinks and an average of 4 residual NHS esters per 8-armed node (5 mM residual NHS esters in the sealant). Mixing of equal volumes of these two macromonomer solutions (the 8-armed macromonomer at 40 mg/mL and the 4-armed macromonomer at 60 mg/mL) provides a sealant having total PEG concentration of 5%, an average crosslinking density of 6 crosslinks and an average of 2 residual NHS esters per 8-armed node (2 mM residual NHS esters in the sealant). Mixing of equal volumes of these two macromonomer solutions (each at 100 mg/mL) provides a sealant having total PEG concentration of 10%, an average crosslinking density of 4 crosslinks and an average of 4 residual NHS esters per 8-armed node (10 mM residual NHS esters in the sealant).

In a second formulation, macromonomer solutions were prepared by dissolving PEG-(NHCO—CH₂CH₂—CONH-linker-O(CO)OSu)₈ (Preparation O) in 0.01 M phosphate, pH 4, and PEG-(NH₂)₄ in 0.1 M phosphate, pH 8.5. Sealants were prepared as described above.

### Example 11

### Gelation Time

Gel time was determined by placing a small magnetic stir bar and the first gel component solution in a 1.5-mL vial, then adding the second component and measuring the time required for the gel to set and stop rotation of the stir bar. For gels formed by triazole formation using a PEG-DBCO and a PEG-azide, the first component was the PEG-DBCO. The concentrations of the gel component solutions were determined by assay in the case of PEG-azide (by UV measurement of the DBCO consumed in the presence of excess DBCO) or by UV absorbance at 308 nm (using e=13,500 M⁻¹ cm⁻¹) in the case of PEG-DBCO. Room temperature was measured as 23° C. Monomer solutions were as follows:

| A | 4-arm PEG₁₀kDa-(DBCO)₄ | | | | |
|---|---|---|---|---|---|
| B | 4-arm PEG₁₀kDa-(NHCO₂—(CH₂)₆—N₃)₄ | | | | |
| C | 4-arm PEG₂₀kDa-(DBCO)₄ | | | | |
| D | 4-arm PEG₂₀kDa-(NH—CO₂—(CH₂)₆—N₃)₄ | | | | |
| Total PEG | temp ° C. | A + B | A + D | C + B | C + D |
| 5% | 23 | 66 sec | 40 | 96 | 64 |
| 10% | 23 | 28 sec | 19 | 36 | 22 |
| 15% | 23 | nd | 13 | 23 | 14 |
| 5% | 25 | 59 | nd | nd | nd |
| 5% | 30 | 46 | nd | nd | nd |
| 5% | 35 | 33 | nd | nd | nd |
| 5% | 37 | 32 | nd | nd | 26 |
| 10% | 37 | 14 | nd | nd | 9 |

For sealants formed using the reaction of a succinimidyl ester or carbonate with an amine to form an amide or carbamate linkage, respectively, the gelation time is a function of the pH of the gel mixture, with gel formation occurring more quickly as the pH increases. For 5% PEG sealants prepared from PEG-(NHCO—CH₂CH₂—CONH-linker-O(CO)OSu)₈ and PEG₅kDa-(NH₂)₄, gel times were measured as 9 sec at pH 9.4 and 72 sec at pH 8.4.

### Example 12

### Sealant Burst Pressure and Swelling Measurement

A device for measuring burst pressure was fabricated from 6061-T6 aluminum. This device allows the application of pressure from air, gas, or liquid buffer to a collagen sausage casing (Weston, 19 mm snack sticks) sealed against a Buna-N O-ring (OD=1 1/16″ (1.066″) 1/16″ width). Pressure is provided by a hand operated 50 to 100 mL syringe and sensed by a NSCDANN100PGUNV gauge pressure sensor (Honeywell). Pressure vs. time data was recorded by a custom computer application written in DAQ Factory (AzeoTech) using a Lab Jack USB DAQ.

Circles approximately 32 mm in diameter were cut from collagen sausage casing (Weston, 19 mm snack sticks). The center of these circles was pierced with the tip of a pasture pipette to give a hole approximately 1.5 mm in diameter. The resulting casings were soaked in PBS. Gels (9 mm diameter×1 mm thick) were then formed over the hole defect using rubber perfusion chamber molds. Test sealants were allowed to cure for 30 minutes then assessed for burst pressure either immediately or after swelling in PBS for 24 h. Sealant swelling ratios were measured by weighing sealant discs immediately after preparation and then following equilibration in PBS for 24 h. Hydrogel sealant properties were compared to previously reported sealants. Comparator A: 10-kDa PEG-(succinimidyl glutarate)₄+10-kDa PEG-(thiol)₄ (1:1 HSE:thiol) at 20% total PEG. Comparator B: 10-kDa PEG-(succinimidyl glutarate)₄+trilysine (1:1 HSE:amine) at 9.4% PEG. PR a-b-c-d series sealants comprised PEG-(linker-HSE)₈+PEG-(amine)₄ wherein a=average mw of the 8-arm PEG, b=average molecular weight of the 4-arm PEG, c =HSE:amine ratio, and d =total % PEG (w/v) in the pre-equilibrium gel mixture. PE x-y-z series sealants comprised PEG-(DBCO-Lys(HSE))₄+PEG-(linker-N₃)₄, wherein x=average mw of the bifunctional PEG, y=average mw of PEG-(linker-N₃)₄, and z=total % PEG (w/v) in the pre-equilibrium gel mixture are shown in the table below.

TABLE 1
Burst pressure and swelling ratios of various NHS-ester
containing hydrogel formulations.

| | Pre-swelling | Post-swelling | |
|---|---|---|---|
| | burst | burst | Swelling |
| Material | pressure (PSI) | pressure (PSI) | ratio |
| Non-adhesive | 0.4 ± 0.3 * | ND | ND |
| Comparator A | 4.9 ± 1.2 | 4.1 ± 0.3 | 3.2 ± 0.2 |
| Comparator B | 2.5 ± 0.8 | 4.2 ± 0.3 | 1.9 ± 0.1 |
| PR 20-2-2-9 | 2.3 ± 0.4 | 2.3 ± 0.2 | 1.7 ± 0.2 |
| PR 20-2-2-13 | 2.3 ± 0.2 | 2.1 ± 0.5 | 2.3 ± 0.1 |
| PR 20-2-2-19 | 3.1 ± 1.1 | 3.9 ± 0.4 | 3.2 ± 0.1 |
| PR 20-2-1.3-13 | 2.8 ± 0.4 | 4.0 ± 0.5 | 1.8 ± 0.1 |
| PR 40-2-2-6 | 1.1 ± 0.4 | 1.2 ± 0.2 | 2.0 ± 0.1 |
| PR 40-2-2-19 | 2.0 ± 0.8 | 2.4 ± 1.2 | 4.6 ± 0.2 |
| PE 20-20-5 | 1.8 ± 0.1 | 1.9 ± 0.4 | 1.7 ± 0.0 |
| PE 20-5-5 | 1.7 ± 0.0 | 1.5 ± 0.5 | 1.3 ± 0.1 |

Burst pressure measurements marked (*) indicate adhesive failure rather than bursting. ND = not determined. The “non-adhesive” control was a tetra-PEG hydrogel containing no NHS ester adhesion groups; burst strength could not be measured as the gel failed to adhere to the collagen.

### Example 13

### Hydrogel Degradation Rates as a Function of R¹

Hydrogels comprising degradable linkers that varied only in R¹ were prepared by mixing solutions of PEG₂₀kDa-(DBCO)₄ and PEG₂₀ kDa-(NH—CO₂—CH(CH₂R¹)(CH₂)₅N₃)₄. A small fraction of a fluorescent erosion probe was added to allow measurement of gel solubilization. Thus, a 50-mg/mL solution of PEG₂₀kDa-(DBCO)₄ (250 uL, 2.50 uMol DBCO end-groups) in water was mixed with 25 μL of a 10-mM solution of the azide-linker-aminoacetylfluorescein (AAF) (0.25 μMol azide) erosion probe in DMF and kept 30 min at ambient temperature. Aliquots (50 uL, 0.42 uMol DBCO) were mixed with 28 uL of 10 mM NaOAc, pH 5.0, followed by 45 uL of 50 mg/mL PEG₂₀kDa-(NH—CO₂—CH(CH₂R¹)(CH₂)₅N₃)₄ (0.42 uMol azide). Gels were formed in 1×9 mm circular diffusion molds. For degradation assays, the gels were suspended in 2 mL of 0.1 M KPᵢ, pH 7.4, at 37° C., and the OD₄₉₃ of the supernatant was periodically measured to monitor fluorescein solubilization. The degelation time T_{dg} was defined as the point at which maximum OD₄₉₃ was observed, indicating complete solubilization of the hydrogel. Results are given in Table 2 and compared with previously reported half-lives for release of acetamidofluorescein (Santi, et al., Proc. Natl. Acad. Sci. USA (2012) 109:6211-6216. It was observed that T_{dg} correlates with the reported half-lives for release of acetamidofluorescein, thus allowing for prediction of the rates of sealant degradation.

TABLE 2
Comparison of hydrogel degelation times at pH 7.4, 37° C. with
previously reported half-lives for release of acetamidofluorescein.

| R¹ | T_{dg} (days) | AAF release t_{1/2} (days) |
|---|---|---|
| 4-chlorophenyl-SO₂ | 1.3 | 1.5 |
| Phenyl-SO₂ | 2.3 | 3.0 |
| Morpholino-SO₂ | 22 | 31 |
| CN | 105 | 100 |

1-26. (canceled)

27. A biodegradable sealant which comprises a biodegradable hydrogel comprised of multivalent polymers, M, coupled to a multiplicity of protein-reactive functional groups,
wherein said protein-reactive functional groups are coupled to the hydrogel through linkers of the formula 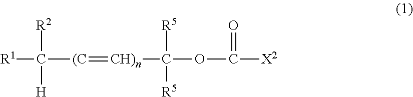
wherein
n is 0 or 1;
at least one or both R¹ and R² is independently CN; NO₂;
optionally substituted aryl;
optionally substituted heteroaryl;
optionally substituted alkenyl;
optionally substituted alkynyl;
COR³ or SOR³ or SO₂R³ wherein
R³ is H or optionally substituted alkyl;
aryl or arylalkyl, each optionally substituted;
heteroaryl or heteroarylalkyl, each optionally substituted; or
OR⁹ or NR⁹₂ wherein each R is independently H or optionally substituted alkyl, or both R⁹ groups taken together with the nitrogen to which they are attached form a heterocyclic ring;
SR⁴ wherein
R⁴ is optionally substituted alkyl;
aryl or arylalkyl, each optionally substituted; or
heteroaryl or heteroarylalkyl, each optionally substituted;
wherein R¹ and R² may be joined to form a 3-8 membered ring; and
wherein one and only one of R¹ and R² may be H or may be alkyl, arylalkyl or heteroarylalkyl, each optionally substituted; and
each R⁵ is independently H or is alkyl, alkenylalkyl, alkynylalkyl, (CH₂CH₂O)ₚ wherein p=1-1000, aryl, arylalkyl, heteroaryl or heteroarylalkyl, each optionally substituted; and
wherein at least one of R¹, R², and R⁵ is substituted with X²,
wherein one and only one of X² is a group that binds to the hydrogel and is not capable of binding to protein unless already bound thereto and the other is a protein-reactive group, P.

28. The biodegradable sealant of claim 27 wherein the hydrogel comprises macromonomers are coupled by crosslinkers of the formula 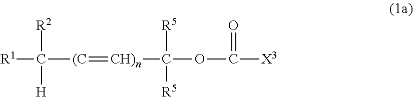
wherein
n is 0 or 1;
wherein X³ is a functional group for binding to the hydrogel and not a protein-reactive group, wherein one of R¹, R², and R⁵ is substituted with X³, and R¹, R² and R⁵ are otherwise as defined in formula (1), and/or
of the formula 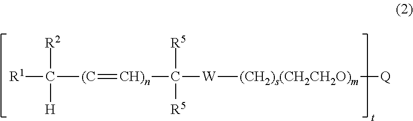
wherein only one of R¹, R² and R⁵ in at least two of the t moieties shown within the bracket comprises said functional group X³ wherein R¹, R² and R⁵ are otherwise as defined in formula (1) and wherein
n is 0 or 1;
m is 0-1,000;
s is 0-2;
t is 2, 4, 8, 16 or 32,
W is O(C═O)O, O(C═O)NH, O(C═O), S, 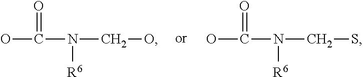
R⁶ is H, alkyl (1-6C), aryl or heteroaryl; and
Q is a core group having a valency=t.

29. The sealant of claim 27 which further comprises one or more drugs coupled to said hydrogel through a biodegradable linker.

30. The sealant of claim 29 wherein said one or more drugs are coupled to the hydrogel through the linker of formula (1b) 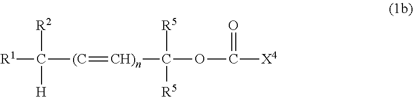
wherein n =0 or 1, and
wherein one of R¹, R² and R⁵ is substituted with X⁴ wherein one X⁴ is a hydrogel binding group and the other is a drug binding group and neither X⁴ is a protein-reactive group unless already coupled to a drug and wherein R¹, R² and R⁵ are otherwise as defined in formula (1).

31. The sealant of claim 28 which further comprises one or more drugs coupled to said hydrogel through a biodegradable linker.

32. The sealant of claim 31 wherein said one or more drugs are coupled to the hydrogel through the linker of formula (1b) 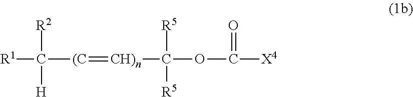
wherein n=0 or 1, and
wherein one of R¹, R² and R⁵ is substituted with X⁴ wherein one X⁴ is a hydrogel binding group and the other is a drug binding group and neither X⁴ is a protein-reactive group unless already coupled to a drug and wherein R¹, R² and R⁵ are otherwise as defined in formula (1).

33. The sealant of claim 28 wherein at least some of the components of said hydrogel are macromonomers containing a multiplicity of groups, X¹ which are reactive with X², X³ or X⁴.

34. The sealant of claim 28 wherein the hydrogel has the formula
(MTₓ)_{y} (3)
wherein M is a macromonomer, T is a crosslinker, x is an integer of 2-40, and y is an integer that results in the formation of a hydrogel,
wherein not all M in said hydrogel need be identical, a plurality of the crosslinkers T couple the y M macromonomers and at least a plurality of T crosslinkers are of the formula (1a) and/or formula (2).

35. The sealant of claim 34 wherein:
at least some M are polyethylene glycol or multi-armed polyethylene glycol and the remainder are polylysine; or
at least some M are polyethylene glycol multi-armed polyethylene glycol and the remainder are polyethylene thiol, or
at least some M are albumin and the remainder are multi-armed polyethylene glycol, or
all of M are multi-armed polyethylene glycol, and/or
wherein the hydrogel or drug binding group X², X³ or X⁴ is selected from the group consisting of thiols or protected thiols, alcohols, acrylates, acrylamides, amines or protected amines, carboxylic acids or protected carboxylic acids, azides, alkynes including cycloalkynes, 1,3-dienes including cyclopentadienes and furans, cyclooctenes, cyclopropenes, alpha-halocarbonyls and 1,2,4,5-tetrazines, and/or
wherein the protein-reactive functional group is a hydroxysuccinimide or sulfohydroxysuccinimide ester or carbonate; a substituted phenyl ester or carbonate; a maleimide, vinylsulfone, or vinylsulfonamide; or an alpha-halo ketone, alpha-halo carboxamide, or alpha-halo carboxylate, an aldehyde, or a perfluorohydrocarbyl group.

36. A method to prepare the sealant of claim 30,
(a) which method comprises reacting a hydrogel with the linker of formula (1b) coupled to a drug and with a linker of formula (1) coupled to said protein-reactive functional group either simultaneously or sequentially, wherein said hydrogel comprises multiple X¹ groups reactive with X² and/or X⁴, or
(b) which method comprises preparing said hydrogel in the presence of a crosslinker of formula (1a) or (2) and a linker of formula (1b) coupled to a drug.

37. A method to prepare the sealant of claim 27,
(a) which method comprises forming said hydrogel in the presence of the linker of formula (1), or
(b) which method comprises reacting said hydrogel with the linker of formula (1) coupled to P.

38. A multilayer hydrogel or multilayer sealant which multilayer comprises at least a first layer and a second layer overlaying the first layer and covalently coupled thereto,
wherein at least two layers in said multilayer have at least one different property.

39. The multilayer hydrogel or multilayer sealant of claim 38 wherein said first and second layers have different degradation rates, or different elastic moduli, or polymer molecular weight or wt % polymer, or comprise different releasable drugs D, or comprise one or more drugs D attached via different releasable linkers, or one layer comprises a releasable drug D while the other does not.

40. The multilayer hydrogel or multilayer sealant of claim 38 wherein said first layer has a pore size with larger average diameter than said second layer.

41. The multilayer hydrogel or multilayer sealant of claim 38 wherein said first and second layers are coupled through excess cognate functional groups employed in forming the hydrogel or sealant.

42. The multilayer hydrogel or multilayer sealant of claim 38 wherein the multilayer is a sealant and said first layer comprises a plurality of protein-reactive functional groups.

43. The multilayer sealant of claim 42 wherein the plurality of protein-reactive functional groups is coupled to said first layer through linkers that are cleavable by an elimination reaction.

44. The multilayer hydrogel or multilayer sealant of claim 38 wherein said drug or drugs is/are coupled to said first layer through a linker that is cleavable by an elimination reaction.

45. The multilayer hydrogel or multilayer sealant of claim 38 wherein at least one of said first layer or second layer or both is a hydrogel crosslinked with at least some crosslinkers that are cleavable by an elimination reaction.

46. The multilayer hydrogel or multilayer sealant of claim 38 which is designed for delivery of drug to tissue which multilayer comprises a first layer adjacent to said tissue having a pore size sufficient to accommodate said drug and a second layer overlaying said first layer and in contact with a biological fluid with which the tissue interacts wherein said second layer has a pore size sufficiently small to prevent entry of proteins from said biological fluid into said first or second layer.
